(19) United States
(12) Patent Application Publication
(10) Pub. No.: US 2011/0065663 A1
(43) Pub. Date: Mar. 17, 2011

(54) ANTI-CANCER COMBINATION THERAPY

(75) Inventors: Jan Balzarini, Heverlee (BE); Sandra Liekens, Begijnendijk (BE)

(73) Assignee: Katholieke Universiteit Leuven, K.U. Leuven R&D

(21) Appl. No.: 12/992,260

(22) PCT Filed: May 15, 2009
(86) PCT No.: PCT/EP09/55955

§ 371 Date: Nov. 11, 2010

(30) Foreign Application Priority Data
May 15, 2008 (GB) 0808841.1
May 30, 2008 (GB) 0809856.8

(51) Int. Cl. A61K 31/7076; A61P 35/00; A61K 31/7072; A61K 31/513; A61K 31/7068

(52) U.S. Cl. 514/46; 514/50; 514/274; 514/49

(57) ABSTRACT

The present invention relates to a combination of therapeutic agents comprising: (a) a cytosine-based anti-cancer drug and/or a purine-based anticancer drug and (b) a therapeutic agent selected from the group consisting of thymidine phosphorylase inhibitors, and antibiotics against Mollicutes bacteria. The present invention also relates to the simultaneous, separate or sequential use of said combination for the treatment of cancer in mammals, especially in humans. The present invention also relates to methods of treatment of cancer, preferably in mammals infected with Mollicutes bacteria.

### FIELD OF THE INVENTION

The present invention relates to a combination of therapeutic agents as a combined preparation for simultaneous, separate or sequential use for the treatment of cancer in mammals, especially in humans. The present invention relates to methods of treatment of cancer in humans infected with Mollicutes bacteria.

### BACKGROUND OF THE INVENTION

Several purine- and pyrimidine-based drugs have been shown to exert anti-cancer activity against a variety of solid tumors and leukemias/lymphomas. Among the pyrimidine-based drugs are the deoxycytidine analogues cytarabine (hereinafter araC), gemcitabine (2′,2′-difluorocytidine) and the preclinical troxacitabine and sapacitabine (the N⁴-palmitoyl prodrug of 2′-cyano-2′-deoxy-araC); the uracil-based 5-fluorouracil (hereinafter FU) and its prodrug capecitabine and ftorafur. Two additional 5-substituted uracil-based nucleoside analogues (e.g. 5-fluoro-dUrd (hereinafter FdUrd) and 5-trifluorothymidine (hereinafter TFT) are not approved yet for clinical use. Among the purine-based analogues are 6-thioguanine (hereinafter 6TG), 6-mercaptopurine (hereinafter 6MP), and its prodrug azathioprine; and the deoxyadenosine analogues fludarabine, cladribine and clofarabine. The deoxyguanosine derivatives nelarabine (which is a water-soluble prodrug of araG), and 2′,2′-difluoroguanosine (hereinafter dFdG) have not yet been formally approved for clinical use. The different cancers that are targeted by these drugs include acute lymphoblastic leukemia, acute myeloid leukemia, chronic lymphocytic leukemia, hairy-cell leukemia, non-Hodgkin lymphoma, pancreatic cancer, lung cancer, breast cancer, bladder cancer, head and neck cancer, renal cancer, skin cancer, prostate cancer, gastrointestinal cancer and colorectal cancer.

It is known that these purine- and pyrimidine-based drugs are highly metabolised by human (often cancer related) metabolising (activating and inactivating) enzymes such as phosphorylases (such as thymidine phosphorylase, hereinafter TP) and kinases (such as thymidine kinase, hereinafter TK).

The eventual cytostatic activity of the antimetabolite cancer drugs highly depends on the balance between activating and inactivating enzymes present in the plasma and the tumor cells. Indeed, mammalian (in particular human) catabolic enzymes such as 5′-nucleotidases (hereinafter 5′-Nu), pyrimidine and purine nucleoside phosphorylases (hereinafter PNP), pyrimidine and purine nucleoside and nucleotide deaminases and nucleotide triphosphatases may prevent efficient conversion of the nucleoside drugs into their active metabolite(s) and thus, may hamper their eventual cytotoxic/anticancer activity. Several reports have clearly demonstrated decreased efficacy of cladribine, fludarabine, araC and gemcitabine in cancer patients due to increased 5′-Nu. It has been found that patients with acute myelocytic leukemia (hereinafter AML) whose blasts express high levels of 5′-Nu, have a worse prognosis than patients with normal 5′-Nu levels. Inactivation of 5FdUrd and TFT is mainly modulated by TP followed by dihydropyrimidine dehydrogenase (hereinafter DPD) that further catabolises 5FU and TF-thymine. Increased DPD expression was found in patients to be related to resistance to 5FU and fluoropyrimidine nucleosides. Cytarabine is broken down into the non-toxic araU by cytidine deaminase, and ara-CMP can be dephosphorylated by cytoplasmic 5′-nucleotidases. Each of the pyrimidine/purine-based drugs displays unique characteristics with regard to its susceptibility to the catabolic versus anabolic enzymes and their molecular mechanisms of drug resistance. Such individual drug properties make them selectively effective against certain types of tumors and ineffective or poorly cytotoxic to other types of tumors and untransformed cells.

Efforts have been devoted to the development of prodrugs of antitumor agents to optimise their pharmacological profile and anticancer activity (i) by circumventing their degradation by catabolic enzymes and/or (ii) by rendering them more tumor-selective and/or by (iii) lowering their toxic side-effects. For example, Capecitabine is an oral formulation of 5FU that can be absorbed from the gastrointestinal tract after which it is metabolized by a cascade of enzymes to 5FU. Recently, a combination of TFT with a potent inhibitor of mammalian TP [5-chloro-6-(1 [2-iminopyrrolidinyl]methyl)uracil hydrochloride (hereinafter TPi)], designated as TAS-102, is under development (Emura T et al. in Int. J. Oncol. (2004) 25:571-8 and EP-1,849,470-A). The mechanism of cytostatic action of TFT is based on inhibition of thymidylate synthase (hereinafter TS) as its monophosphate and incorporation of the drug into the DNA after conversion to its triphosphate metabolite (Emura T et al. Int J Mol Med 2004; 13:249-55). However, TFT is rapidly inactivated by human TP, which converts TFT to its inactive base. Therefore, a new drug formulation containing TFT and TPi is being developed. At present, TAS-102 is being evaluated in phase I clinical trials for the treatment of various solid tumors. Thus, TP has an ambiguous role in fluoropyrimidine-based chemotherapy. It may enhance the anti-tumoral properties of 5FU prodrugs such as capecitabine on the one hand, but it may inactivate pyrimidine 2′-deoxyuridine derivatives, such as TFT, on the other hand. However, there is still a great need for more potent anti-cancer treatments or anti-cancer treatments with less side-effects.

### SUMMARY OF THE INVENTION

The present inventors have surprisingly found that the combination of at least one thymidine phosphorylase inhibitor (hereinafter TPI) combined with at least one cytosine-based anticancer drug or with at least one purine-based anticancer drug, restore the cytotoxicity of these drugs, when used against cancer, in particular against cancer in a mammal infected with Mollicutes bacteria. Said combination is useful for the treatment of cancer in a mammal, preferably when said mammal is infected with Mollicutes bacteria selected from the group consisting of Mycoplasma sp., Acheloplasma sp., Ureaplasma sp., Phytoplasma sp. and Spiroplasma sp.

Indeed, both cytosine- and purine-based anticancer drug, are drugs that are not expected to be substrates for TP because they belong to two entirely different classes of compounds for which so far, it has never been shown that they are sensitive to the degradation by TP. The TP enzyme has been shown to selectively act on thymidine and deoxyuridine analogues.

Accordingly a first aspect of the present invention relates to a combination of therapeutic agents comprising: (a) a cytosine-based anti-cancer drug and/or a purine-based anticancer drug and (b) a therapeutic agent selected from the group consisting of thymidine phosphorylase inhibitors, and antibiotics against Mollicutes bacteria. The present invention also relates to a composition comprising (a) at least one cytosine-based anti-cancer drug and/or purine-based anticancer drug and (b) at least one therapeutic agent selected from the group consisting of thymidine phosphorylase inhibitors, and antibiotics against Mollicutes bacteria.

A second aspect of the present invention relates to a pharmaceutical composition comprising or consisting of one or more pharmaceutically acceptable carriers or excipients together with the above-defined combination of therapeutic agents as active ingredients.

A further aspect of the present invention relates to said combination or composition for use in the treatment of cancer in a mammal, preferably in the treatment of cancer in a mammal infected with Mollicutes bacteria. The present invention also relates to the use of said combination for the preparation of a medicament for the treatment of cancer in a mammal, preferably for the treatment of cancer in a mammal infected with Mollicutes bacteria. Said combination can be used in said treatment by consecutive administration, wherein said therapeutic agent (b) is administered prior to said cytosine- or purine-based anticancer drug. Preferably, said therapeutic agent (b) is administered from 1 to 4 days prior to said cytosine- or purine-based anticancer drug (a). In an embodiment, said Mollicutes bacteria are selected from the group consisting of Mycoplasma sp., Acheloplasma sp., Ureaplasma sp., Phytoplasma sp. and Spiroplasma sp.

One embodiment of the present invention concerns combinations wherein said cytosine-based anti-cancer drug is selected from the group consisting of cytarabine, gemcitabine, troxacitabine, sapacitabine.

One embodiment of the present invention concerns combinations wherein said purine based anti-cancer drug is selected from 6-thioguanine, 6-mercaptopurine, azathioprine, 2-chloroadenine, 2-fluoroadenine, nelarabine, 2′,2′-difluoroguanosine, 9-β-D-arabinosylguanine (araG), clofarabine, cladribine, 6-methyl-purineriboside (6-methyl-purine-beta-D-riboside or 6-methyl-purine-alpha-D-riboside), and fludarabine.

One embodiment of the present invention concerns combinations wherein said therapeutic agent (b) is an uracil derivative, a solvate or a pharmaceutically acceptable salt thereof, said uracil derivative being represented by the structural formula (I)

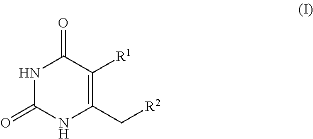

wherein:
R¹ is selected from chloro, bromo, iodo, cyano or C₁₋₄alkyl;
R² is a 4-8 membered heterocyclic group having 1, 2 or 3 nitrogen atoms, optionally substituted by one or more substituents independently selected from the group consisting of C₁₋₄alkyl, imino, hydroxyl, hydroxymethyl, methanesulfonyloxy, amino and nitro; or R² is an amidinothio group, the nitrogen atoms of which may each be independently substituted by C₁₋₄alkyl; or R² is a guanidino group, the nitrogen atoms of which may each be independently substituted by C₁₋₄alkyl or cyano; or R² is C₁₋₄alkyl-amidino; or R² is amino, mono-C₁₋₄alkylamino or di-C₁₋₄alkylamino; or R² is a group with the structural formula—CH₂N(R^{a})R^{b} wherein R^{a} and R^{b} are independently hydrogen or C₁₋₄alkyl or R^{a} and R^{b} may form a pyrrolidine ring together with the nitrogen atom to which they are bonded; or R² is a group with the structural formula—NH—(CH₂)ₘ—Z wherein Z is cyano, amino, mono-C₁₋₄alkylamino or di-C₁₋₄alkylamino, and m is an integer from 0 to 3; or R² is a group with the structural formula NR^{c}(CH₂)ₙ—OH in which R^{c} is hydrogen or C₁₋₄alkyl, and n is an integer from 1 to 4; or R² is a group with the structural formula—X—Y in which X is S or NH, and Y is selected from the group consisting of 2-imidazolin-2-yl, 2-imidazolyl, 1-methylimidazol-2-yl, 1,2,4-triazol-3-yl, 2-pyrimidyl and 2-benzimidazolyl group; or R² is a ureido or thioureido group, the nitrogen atoms of which may each be independently substituted by C₁₋₄alkyl. One embodiment of the present invention concerns combinations wherein in said structural formula (I) R² is selected from the group consisting of 2-iminopyrrolidin-1-yl, 1-azetidinyl, 1-pyrrolidinyl, 2-pyrrolin-1-yl, 3-pyrrolin-1-yl, 1-pyrrolyl, 1-pyrazolidinyl, 2-pyrazolin-1-yl, 3-pyrazolin-1-yl, 4-pyrazolin-1-yl, 1-pyrazolyl, 1-imidazolidinyl, 2-imidazolin-1-yl, 3-imidazolin-1-yl, 4-imidazolin-1-yl, 1-imidazolyl, 1,2,3-triazol-1-yl, 1,2,4-triazol-1-yl, piperidino, 1-piperazyl, morpholino, 1-perhydroazepinyl, 1-perhydroazocinyl, amidino-thio, N-methylamidinothio, N,N′-dimethylamidinothio, 1-guanidino, 1-methylguanidino, 3-methylguanidino, 2,3-dimethylguanidino, 2-cyano-3-methylguanidino, acetoamidino, N-methylamino, N,N-dimethylamino, N-ethylamino, N,N-diethylamino, N-propylamino, N-isopropylamino, N-methylaminomethyl, N,N-dimethylaminomethyl, 1-pyrrolidinylmethyl, N,N-dimethylhydrazino, N-(2-aminoethyl)amino, N-(2-(N,N-dimethyl)amino-ethyl)amino, N-(3-aminopropyl)amino, N-(2-cyanoethyl)amino, N-(2-hydroxyethyl)-N-methylamino, N-(3-hydroxypropyl)amino, N-(4-hydroxy-butyl)amino, 2-imidazolin-2-thio, 2-imidazolin-2-amino, imidazol-2-thio, 1-methylimidazol-2-thio, 1,2,4-triazol-3-thio, pyrimidin-2-thio, benzimidazol-2-thio and 3-methylthioureido. Preferably, R² is 2-iminopyrrolidin-1-yl. One embodiment of the present invention concerns combinations wherein in said structural formula (I), R¹ is bromo, cyano or methyl.

Preferably, said uracil derivative, a solvate or a pharmaceutically acceptable salt thereof is selected from the group consisting of 5-chloro-6-(1-[2-imino-pyrrolidinyl]methyl)uracil hydrochloride, 6-imidazolylmethyl-5-fluorouracil, 5-chloro-6-(1-pyrrolidinylmethy)uracil, 5-bromo-6-(1-pyrrolidinylmethyl)uracil, 5-chloro-6-(1-azetidinylmethyl)-uracil, 5-bromo-6-(1-(2-iminopyrrolidinyl)methyl)uracil hydrochloride, 5-cyano-6-(1-(2-iminopyrrolidinyl)methyl)uracil, 5-chloro-6-(1-(2-imino-imidazolidinyl)methyl)uracil, 5-bromo-6-(1-(2-iminoimidazolidinyl)-methyl)uracil, 5-chloro-6-(1-imidazolylmethyl)uracil hydrochloride, 2-(5-chlorouracil-6-ylmethyl)isothiourea hydrochloride, 2-(5-cyanouracil-6-ylmethyl)isothiourea hydrochloride and 5-chloro-6-(1-guanidino)methyl-uracil hydrochloride. Preferably said uracil derivative is 5-chloro-6-(1-[2-imino-pyrrolidinyl]methyl)uracil hydrochloride, 6-imidazolylmethyl-5-fluorouracil or 6-imidazolylmethyl-5-chlorouracil. More preferably said uracil derivative is 5-chloro-6-(1-[2-imino-pyrrolidinyl]methyl)uracil hydrochloride.

One embodiment of the present invention concerns combinations wherein said therapeutic agent (b) is selected from the group consisting of thymidine phosphorylase inhibitors, and wherein the molar ratio between said cytosine or purine-based anti-cancer drug (a) and said therapeutic agent (b) ranges from 25:1 to 0.01:1.

One embodiment of the present invention concerns combinations wherein said cytosine-based anti-cancer drug is selected from the group consisting of cytarabine, gemcitabine, troxacitabine, sapacitabine and said thymidine phosphorylase inhibitor is selected from the group comprising 5-chloro-6-(1 [2-imino-pyrrolidinyl]methyl)uracil hydrochloride, 6-imidazolylmethyl-5-fluorouracil, 5-chloro-6-(1-pyrrolidinylmethy)uracil, 5-bromo-6-(1-pyrrolidinylmethyl)uracil, 5-chloro-6-(1-azetidinylmethyl)-uracil, 5-bromo-6-(1-(2-iminopyrrolidinyl)methyl)uracil hydrochloride, 5-cyano-6-(1-(2-iminopyrrolidinyl)methyl)uracil, 5-chloro-6-(1-(2-imino-imidazolidinyl)methyl)uracil, 5-bromo-6-(1-(2-iminoimidazolidinyl)-methyl)uracil, 5-chloro-6-(1-imidazolylmethyl)uracil hydrochloride, 2-(5-chlorouracil-6-ylmethyl)isothiourea hydrochloride, 2-(5-cyanouracil-6-ylmethyl)isothiourea hydrochloride and 5-chloro-6-(1-guanidino)methyl-uracil hydrochloride. Preferably, said cytosine-based anti-cancer drug is selected from the group consisting of cytarabine, gemcitabine, and troxacitabine, and said thymidine phosphorylase inhibitor is selected from the group comprising 5-chloro-6-(1-[2-imino-pyrrolidinyl]methyl)uracil hydrochloride, 6-imidazolylmethyl-5-fluorouracil, 5-chloro-6-(1-pyrrolidinylmethy)uracil, 5-bromo-6-(1-pyrrolidinylmethyl)uracil, 5-chloro-6-(1-azetidinylmethyl)-uracil, 5-bromo-6-(1-(2-iminopyrrolidinyl)methyl)uracil hydrochloride, 5-cyano-6-(1-(2-iminopyrrolidinyl)methyl)uracil, 5-chloro-6-(1-(2-imino-imidazolidinyl)methyl) uracil, 5-bromo-6-(1-(2-iminoimidazolidinyl)-methyl)uracil, 5-chloro-6-(1-imidazolylmethyl)uracil hydrochloride, 2-(5-chlorouracil-6-ylmethyl)isothiourea hydrochloride, 2-(5-cyanouracil-6-ylmethyl)isothiourea hydrochloride and 5-chloro-6-(1-guanidino)methyl-uracil hydrochloride. Preferably, said cytosine-based anti-cancer drug is cytarabine, or gemcitabine, and said thymidine phosphorylase inhibitor is selected from the group comprising 5-chloro-6-(1 [2-imino-pyrrolidinyl]methyl)uracil hydrochloride, 6-imidazolylmethyl-5-fluorouracil, 5-chloro-6-(1-pyrrolidinylmethy)uracil, 5-bromo-6-(1-pyrrolidinylmethyl)uracil, 5-chloro-6-(1-azetidinylmethyl)-uracil, 5-bromo-6-(1-(2-iminopyrrolidinyl)methyl)uracil hydrochloride, 5-cyano-6-(1-(2-iminopyrrolidinyl)methyl)uracil, 5-chloro-6-(1-(2-imino-imidazolidinyl)methyl)uracil, 5-bromo-6-(1-(2-iminoimidazolidinyl)-methyl)uracil, 5-chloro-6-(1-imidazolylmethyl)uracil hydrochloride, 2-(5-chlorouracil-6-ylmethyl)isothiourea hydrochloride, 2-(5-cyanouracil-6-ylmethyl)isothiourea hydrochloride and 5-chloro-6-(1-guanidino)methyl-uracil hydrochloride.

One embodiment of the present invention concerns combinations wherein said purine based anti-cancer drug is selected from 6-thioguanine, 6-mercaptopurine, azathioprine, 2-chloroadenine, 2-fluoroadenine, nelarabine, 2′,2′-difluoroguanosine, 9-β-D-arabinosylguanine (araG), clofarabine, cladribine, 6-methyl-purineriboside (6-methyl-purine-beta-D-riboside or 6-methyl-purine-alpha-D-riboside), and fludarabine and said thymidine phosphorylase inhibitor is selected from the group comprising 5-chloro-6-(1-[2-imino-pyrrolidinyl]methyl)uracil hydrochloride, 6-imidazolylmethyl-5-fluorouracil, 5-chloro-6-(1-pyrrolidinylmethy)uracil, 5-bromo-6-(1-pyrrolidinylmethyl)uracil, 5-chloro-6-(1-azetidinylmethyl)-uracil, 5-bromo-6-(1-(2-iminopyrrolidinyl)methyl)uracil hydrochloride, 5-cyano-6-(1-(2-iminopyrrolidinyl)methyl)uracil, 5-chloro-6-(1-(2-imino-imidazolidinyl)methyl) uracil, 5-bromo-6-(1-(2-iminoimidazolidinyl)-methyl)uracil, 5-chloro-6-(1-imidazolylmethyl)uracil hydrochloride, 2-(5-chlorouracil-6-ylmethyl)isothiourea hydrochloride, 2-(5-cyanouracil-6-ylmethyl)isothiourea hydrochloride and 5-chloro-6-(1-guanidino)methyl-uracil hydrochloride. Preferably, said purine based anti-cancer drug is selected from azathioprine, 2-chloroadenine, 2-fluoroadenine, nelarabine, 2′,2′-difluoroguanosine, 9-β-D-arabinosylguanine (araG), clofarabine, cladribine, 6-methyl-purineriboside and fludarabine, and said thymidine phosphorylase inhibitor is selected from the group comprising 5-chloro-6-(1 [2-imino-pyrrolidinyl]methyl)uracil hydrochloride, 6-imidazolylmethyl-5-fluorouracil, 5-chloro-6-(1-pyrrolidinylmethy)uracil, 5-bromo-6-(1-pyrrolidinylmethyl)uracil, 5-chloro-6-(1-azetidinylmethyl)-uracil, 5-bromo-6-(1-(2-iminopyrrolidinyl)methyl)uracil hydrochloride, 5-cyano-6-(1-(2-iminopyrrolidinyl)methyl)uracil, 5-chloro-6-(1-(2-imino-imidazolidinyl)methyl)uracil, 5-bromo-6-(1-(2-iminoimidazolidinyl)-methyl)uracil, 5-chloro-6-(1-imidazolylmethyl)uracil hydrochloride, 2-(5-chlorouracil-6-ylmethyl)isothiourea hydrochloride, 2-(5-cyanouracil-6-ylmethyl)isothiourea hydrochloride and 5-chloro-6-(1-guanidino)methyl-uracil hydrochloride. Preferably, said purine based anti-cancer drug is selected from azathioprine, nelarabine, 9-β-D-arabinosylguanine (araG), clofarabine, cladribine, 6-methyl-purineriboside and fludarabine, and said thymidine phosphorylase inhibitor is selected from the group comprising 5-chloro-6-(1 [2-imino-pyrrolidinyl]methyl)uracil hydrochloride, 6-imidazolylmethyl-5-fluorouracil, 5-chloro-6-(1-pyrrolidinylmethy)uracil, 5-bromo-6-(1-pyrrolidinylmethyl)uracil, 5-chloro-6-(1-azetidinylmethyl)-uracil, 5-bromo-6-(1-(2-iminopyrrolidinyl)methyl)uracil hydrochloride, 5-cyano-6-(1-(2-iminopyrrolidinyl)methyl)uracil, 5-chloro-6-(1-(2-imino-imidazolidinyl)methyl)uracil, 5-bromo-6-(1-(2-iminoimidazolidinyl)-methyl)uracil, 5-chloro-6-(1-imidazolylmethyl)uracil hydrochloride, 2-(5-chlorouracil-6-ylmethyl)isothiourea hydrochloride, 2-(5-cyanouracil-6-ylmethyl)isothiourea hydrochloride and 5-chloro-6-(1-guanidino)methyl-uracil hydrochloride.

One embodiment of the present invention concerns combinations comprising 5-chloro-6-(1-[2-imino-pyrrolidinyl]methyl)uracil hydrochloride with a cytosine- or purine-based anti-cancer drug (a) selected from the group consisting of cytarabine, gemcitabine, troxacitabine, sapacitabine, 6-thioguanine, 6-mercaptopurine, azathioprine, nelarabine, 2-chloroadenine, 2-fluoroadenine, 2′,2′-difluoroguanosine, 9-β-D-arabinosylguanine (araG), clofarabine, cladribine, 6-methyl-purineriboside, and fludarabine.

The antibiotic against Mollicutes may be selected from (i) macrolide antibiotics, (more in particular erythromycin, azithromycin or clarithromycin), (ii) tetracyclines (more in particular doxycycline or minocycline) and (iii) fluoroquinolones (more in particular ciprofloxacin or levofloxacin). In another embodiment of the present invention, the antibiotic may be selected from antibiotics active (e.g. with IC₅₀<100 μg/ml) against at least one of Phytoplasma, Ureaplasma, Entomoplasma, Anaeroplasma, Spiroplasma, Mycoplasma mycoides, Mycoplasma pirum, Mycoplasma orale, Mycoplasma arginini, Mycoplasma genitalium, Mycoplasma hominis, Acholeplasma laidlawii, Mycoplasma penetrans, Mycoplasma fermentans, Mycoplasma pneumoniae, Mycoplasma ovipneumoniae, Mycoplasma hiopneumoniae or Mycoplasma hyorhinis.

One embodiment of the present invention concerns combinations, wherein said antibiotic against Mollicutes bacteria is a Mycoplasma-specific antibiotic.

Preferably, said antibiotic against Mollicutes bacteria is selected from the group consisting of plasmocin; herbicolin A; tetracyclines including doxycycline or minocycline; (fluoro)quinolones including ciprofloxacin, enrofloxacin, gemifloxacin or levofloxacin; macrolides including azithromycin, erythromycin or clarithromycin; and linkomycin.

One embodiment of the present invention concerns combinations, wherein the molar ratio between said cytosine- or purine-based anti-cancer drug and said antibiotic against Mollicutes bacteria ranges from 10:1 to 0.01:1.

One embodiment of the present invention concerns combinations wherein said cytosine-based anti-cancer drug is selected from the group consisting of cytarabine, gemcitabine, troxacitabine, sapacitabine and said Mollicutes antibiotic is selected from plasmocin; herbicolin A; tetracyclines including doxycycline or minocycline; (fluoro)quinolones including ciprofloxacin, enrofloxacin, gemifloxacin or levofloxacin; macrolides including azithromycin, erythromycin or clarithromycin; and linkomycin. Preferably said cytosine-based anti-cancer drug is selected from the group consisting of cytarabine, gemcitabine, or troxacitabine and said Mollicutes antibiotic is selected from the group comprising plasmocin; herbicolin A; doxycycline, minocycline; ciprofloxacin, enrofloxacin, gemifloxacin, levofloxacin; azithromycin, erythromycin, clarithromycin; and linkomycin. More preferably said cytosine-based anti-cancer drug is cytarabine or gemcitabine and said Mollicutes antibiotic is selected from the group comprising plasmocin; herbicolin A; doxycycline, minocycline; ciprofloxacin, enrofloxacin, gemifloxacin, levofloxacin; azithromycin, erythromycin, clarithromycin; and linkomycin.

One embodiment of the present invention concerns combinations wherein said purine based anti-cancer drug is selected from 6-thioguanine, 6-mercaptopurine, azathioprine, 2-chloroadenine, 2-fluoroadenine, nelarabine, 2′,2′-difluoroguanosine, 9-β-D-arabinosylguanine (araG), clofarabine, cladribine, 6-methyl-purineriboside (6-methyl-purine-beta-D-riboside or 6-methyl-purine-alpha-D-riboside), and fludarabine, and said Mollicutes antibiotic is selected from plasmocin; herbicolin A; tetracyclines including doxycycline or minocycline; (fluoro)quinolones including ciprofloxacin, enrofloxacin, gemifloxacin or levofloxacin; macrolides including azithromycin, erythromycin or clarithromycin; and linkomycin. Preferably, said purine based anti-cancer drug is selected from azathioprine, 2-chloroadenine, 2-fluoroadenine, nelarabine, 2′,2′-difluoroguanosine, 9-β-D-arabinosylguanine (araG), clofarabine, cladribine, 6-methyl-purineriboside and fludarabine, and said Mollicutes antibiotic is selected from the group comprising plasmocin; herbicolin A; doxycycline, minocycline; ciprofloxacin, enrofloxacin, gemifloxacin, levofloxacin; azithromycin, erythromycin, clarithromycin; and linkomycin. Preferably, said purine based anti-cancer drug is selected from azathioprine, nelarabine, 9-β-D-arabinosylguanine (araG), clofarabine, cladribine, 6-methyl-purineriboside and fludarabine, and said Mollicutes antibiotic is selected from the group comprising plasmocin; herbicolin A; doxycycline, minocycline; ciprofloxacin, enrofloxacin, gemifloxacin, levofloxacin; azithromycin, erythromycin, clarithromycin; and linkomycin.

One embodiment of the present invention concerns combinations comprising plasmocin with a cytosine- or purine-based anti-cancer drug (a) selected from the group consisting of cytarabine, gemcitabine, troxacitabine, sapacitabine, 6-thioguanine, 6-mercaptopurine, azathioprine, nelarabine, 2-chloroadenine, 2-fluoroadenine, 2′,2′-difluoroguanosine, 9-β-D-arabinosylguanine (araG), clofarabine, cladribine, 6-methyl-purineriboside, and fludarabine.

Yet another aspect of the present invention relates to a method for the prevention or treatment of cancer in an animal (more particularly a mammal or a human), wherein an therapeutically effective amount of the above-defined combination of therapeutic agents, optionally together with one or more pharmaceutically acceptable carriers in the form of a pharmaceutical composition is provided and/or administered to said animal in need thereof. In a particular embodiment of this method, the anti-cancer drug (a) and the inhibitor or antibiotic (b) are administered simultaneously to the animal. In another particular embodiment of the method, the anti-cancer drug (a) and the inhibitor or antibiotic (b) are administered sequentially to the animal, the inhibitor or antibiotic (b) being preferably administered a substantial period of time before the anti-cancer drug (a).

### DESCRIPTION OF THE FIGURES

FIG. 1A represents a picture showing the PCR analysis for M. hyorhinis in cell extracts of MCF-7 and MCF-7/HYOR. Lane 1 shows the non-template control; lane 2 shows the uninfected MCF-7 extract; lane 3 shows the infected MCF-7/HYOR extract.

FIG. 1B represents pictures representing DNA staining with Hoechst 33342 in control MCF-7 (a), MCF-7/HYOR (b) and MCF-7/HYOR cells treated with 10 μM TPi (c). Arrows indicate the presence of nucleic acid-rich particles in the cytosol.

FIG. 2 shows Western Blot analysis using a polyclonal antibody against human TP. A band of 55 kDa could be detected in cell lysates of MCF-7 that were transfected with the human TP gene. No human TP was detected in cell extracts of MCF-7 or mycoplasma-infected MCF-7/HYOR cells.

FIG. 3 represents a graph showing the time-course of the conversion of dThd to thymine by M. hyorhinis-infected MCF-7 cell culture supernatants. The medium of MCF-7 and MCF-7/HYOR cells was incubated with 200 μM dThd at 37° C. At different time points, aliquots were withdrawn and the conversion of dThd into thymine was quantified by HPLC analysis. As a positive control 0.025 U of recombinant E. coli TP were used. In one assay, the medium of MCF-7/HYOR cells was filtered through a 0.1 μm syringe filter. In contrast to MCF-7/HYOR cells, no conversion of dThd was observed in the medium of MCF-7 cells, MCF-7/HYOR cell cultures treated with TPi or filtered medium of MCF-7/HYOR cells. Values are the means of 3 separate experiments±S.E.M.

FIG. 4 represents a graph showing the incorporation of dThd, thymine, 2′-deoxyuridine and fluoropyrimidine nucleoside analogues into nucleic acids in the presence or absence of 10 μM TPi. MCF-7 and MCF-7/HYOR cells were incubated overnight with 1 μCi of radiolabeled compound. The next day, the amount of radioactive compound that was incorporated into the nucleic acids was measured. Values are presented as means±S.E.M. of at least three independent experiments. *p<0.01 compared to control MCF-7 cells.

FIG. 5 represents a graph showing the comparison of gemcitabine metabolite distribution in MCF-7 and MCF-7/HYOR cells. MCF-7 and MCF-7/HYOR cells were incubated for 24 h with 1 μCi of radiolabeled gemcitabine (dFdC). The distribution of the different metabolites was determined by HPLC analysis. dFdCMP: gemcitabine monophosphate; dFdCDP: gemcitabine diphosphate; dFdCTP: gemcitabine triphosphate.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

When describing the invention, the terms used are to be construed in accordance with the following definitions, unless a context dictates otherwise.

Abbreviations: BrdUrd: 5-bromo-2′-deoxyuridine; CIdUrd: 5-chloro-2′-deoxyuridine; 5′DFUR: 5-fluoro-5′-deoxyuridine; DPD: dihydropyrimidine dehydrogenase; dThd: thymidine; dUrd: 2′-deoxyuridine; FdUMP: 5-fluoro-2′-deoxyuridine-5′-monophosphate; FdUrd: 5-fluoro-2′-deoxyuridine; 5FU: 5-fluorouracil; IC₅₀: 50% inhibitory concentration; IdUrd: 5-iodo-2′-deoxyuridine; MCF-7/HYOR: MCF-7 cells infected with M. hyorhinis; PD-ECGF: platelet-derived endothelial cell growth factor; TFT: 5-trifluorothymidine; Thy: thymine; TK: thymidine kinase; TP: thymidine phosphorylase; TPI: thymidine phosphorylase inhibitor; TPi: 5-chloro-6-(1 [2-iminopyrrolidinyl]methyl)uracil hydrochloride; TS: thymidylate synthase; Ura: uracil.

The term “nucleoside-based anti-cancer drug” as used herein refers to anti-cancer drugs (anti-cancer agents whether or not at this moment officially approved for human use) which comprise a purine or pyrimidine structure and interfere with nucleoside, nucleotide, DNA or RNA synthesis, repair or necessary changes for having a proliferation of the cell. They can be divided into purine- or pyrimidine-based anti-cancer drugs.

The term “cytosine-based anti-cancer drug” as used herein refers to anti-cancer drugs which comprise an optionally substituted 4-amino-pyrimidine-2-one structure and interfere with nucleoside, nucleotide, DNA or RNA synthesis, repair or necessary changes for having a proliferation of the cell. Preferably said cytosine based anticancer drug is a cytidine derivative such a cytidine stereoisomer, halogenated cytidine, halogenated deoxycytidine, cyano derivative thereof, alkylcarbonyl derivative thereof and the like. Non limiting example of suitable cytosine-based anticancer drug comprises cytarabine, gemcitabine, troxacitabine, or sapacitabine.

The term “purine-based anti-cancer drug” as used herein refers to anti-cancer drugs which comprise a purine structure and interfere with nucleoside, nucleotide, DNA or RNA synthesis, repair or necessary changes for having a proliferation of the cell, such as azathioprine, fludarabine, chlofarabine, cladribine, nelarabine, 2,2difluorodeoxyguanosine, 2-chloroadenine and 2-fluoroadenine and the like.

The term “inhibitor of a nucleoside metabolising enzyme” as used herein refers to compounds or drugs (whether or not at this moment officially approved for human use) which inhibit enzymes responsible for the degradation of nucleosides.

The term “antibiotic against Mollicutes bacteria” as used herein refers to antibiotics (anti-bacterial agents whether or not at this moment officially approved for human use) which have a MIC below 100 μg/ml against at least one Mollicutes, e.g. one mycoplasma, species.

The term “C₁₋₄alkyl” as a group or part of a group refers to a hydrocarbyl radical of Formula CₙH₂ₙ₊₁ wherein n is a number ranging from 1 to 4. Generally, alkyl groups of this invention comprise from 1 to 4 carbon atoms, preferably from 1 to 3 carbon atoms, more preferably 1 to 2 carbon atoms. Alkyl groups may be linear or branched and may be substituted as indicated herein. Thus, for example, C₁₋₄alkyl includes for example methyl, ethyl, n-propyl, i-propyl, 2-methyl-ethyl, butyl and its isomers (e.g. n-butyl, i-butyl and tert-butyl) and the like.

The term “amidinothio” as a group or part of a group, refers to a group of Formula

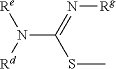

wherein R^{d}, R^{e} and R^{g} are each independently selected from hydrogen or C₁₋₄alkyl.

The term “guanidino” as a group or part of a group, refers to a group of Formula

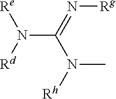

wherein R^{d}, R^{e}, R^{g} and R^{h} are each independently selected from hydrogen or C₁₋₄alkyl.

The term “imino” as a group or part of a group refers to the group NH═.

The term “C₁₋₄alkyl-amidino” as a group or part of a group, refers to a group of Formula

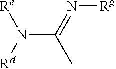

wherein R^{d}, R^{e} and R^{g} are each independently selected from hydrogen or C₁₋₄alkyl, wherein at least one R^{d}, R^{e} or R^{g} is C₁₋₄alkyl as defined herein.

The term “mono- or di-C₁₋₆alkylamino” as a group or part of a group, refers to a group of Formula—N(R^{d})R^{e} wherein R^{d} and R^{e} are each independently selected from hydrogen or C₁₋₄alkyl, wherein at least one R^{d} or R^{e} is C₁₋₄alkyl as defined herein.

The term “ureido” as a group or part of a group, refers to a group of Formula NR^{h}—CO—N(R^{d})R^{e} wherein R^{d}, R^{e} and R^{h} are each independently selected from hydrogen or C₁₋₄alkyl.

The term “thioureido” as a group or part of a group, refers to a group of Formula NR^{h}—CS—N(R^{d})R^{e} wherein R^{d}, R^{e} and R^{h} are each independently selected from hydrogen or C₁₋₄alkyl.

As used in the specification and the appended claims, the singular forms “a”, “an,” and “the” include plural referents unless the context clearly dictates otherwise. By way of example, “a cytosine-based anti-cancer drug” means one cytosine-based anti-cancer drug or more than one cytosine-based anti-cancer drug, i.e. it refers to “at least one cytosine-based anti-cancer drug”.

The terms described above and others used in the specification are well understood to those in the art.

Embodiments of this invention are now set forth.

In an embodiment, the present invention provides a combination of therapeutic agents comprising at least (a) a nucleoside-based anti-cancer drug and (b) an agent selected from an inhibitor of a nucleoside metabolising enzyme and an antibiotic against Mollicutes bacteria.

The present invention also provides a pharmaceutical composition comprising or consisting of one or more pharmaceutically acceptable carriers or excipients together with the above-defined combination of therapeutic agents as active ingredients.

The present invention also provides a method for the prevention or treatment of cancer in an animal (more particularly a mammal or a human), wherein an effective amount of the above-defined combination of therapeutic agents, optionally together with one or more pharmaceutically acceptable carriers in the form of a pharmaceutical composition is provided and/or administered to said animal in need thereof. In a particular embodiment of this method, the anti-cancer drug (a) and the inhibitor or antibiotic (b) are administered simultaneously to the animal. In another particular embodiment of the method, the anti-cancer drug (a) and the inhibitor or antibiotic (b) are administered sequentially to the animal, the inhibitor or antibiotic (b) being preferably administered a substantial period of time before the anti-cancer drug (a).

The combination of therapeutic agents, and the pharmaceutical compositions, of the present invention do not comprise nucleoside-based anti-cancer drugs (a) that require mammalian (e.g. human) cellular enzymes such as pyrimidine nucleoside phosphorylases, nucleotidases, purine nucleoside phosphorylases or deaminases, to be activated, such as is the case for capecitabine, 5-fluoro-5′-deoxyuridine (5′DFUR) or ftorafur.

In a particular embodiment of the different aspects of the invention, the pharmaceutical composition is a combined preparation for simultaneous, separate or sequential use for the treatment or prevention of cancer (including tumor formation, growth and/or metastasis).

In another embodiment of the different aspects of the present invention, the nucleoside-based anti-cancer drugs (a) may be selected from (i) pyrimidine-based anti-cancer drugs (i.e. comprising a pyrimidine structural moiety, such as 5-fluorouracil and cytosine) and (ii) purine-based anti-cancer drugs (i.e. comprising a purine structural moiety, such as azathioprine, 2-chloroadenine and 2-fluoroadenine).

In a more particular embodiment, the purine-based anti-cancer drugs (a) useful in this invention may be selected from adenine derivatives (comprising a substituted or non-substituted 6-amino-purine structure) and guanine derivatives (comprising a substituted or non-substituted 2-amino-purin-6-one structure). In a more particular embodiment, the purine-based anti-cancer drugs (a) may be selected from mercaptopurine (6 MP), thioguanine (6TG), azathioprine, fludarabine, cladribine, clofarabine, 9-β-D-arabinosylguanine (araG) and 2′,2′-difluoroguanosine (dFdG).

In another more particular embodiment, the pyrimidine-based anti-cancer drugs (a) useful in this invention may be selected from thymine derivatives (comprising a substituted or non-substituted 5-methylpyrimidine-2,4-dione structure), cytosine derivatives (comprising a substituted or non-substituted 4-amino-pyrimidine-2-one structure) and uracil derivatives (comprising a substituted or non-substituted pyrimidine-2,4-dione structure). In another more particular embodiment, the pyrimidine-based anti-cancer drugs (a) useful in this invention may be selected from cytarabine (araC), gemcitabine (dFdC), 5-fluorouracil (FU), 5-fluoro-2′-deoxyuridine (5FdUrd) and 5-trifluorothymidine (TFT).

In another embodiment of the aspects of the present invention, the inhibitor (b) of a nucleoside metabolising enzyme may be selected from (i) pyrimidine nucleoside phosphorylase inhibitors such as TP inhibitors (hereinafter TPI) and uridine phosphorylase (UP) inhibitors; (ii) nucleotidase inhibitors (more in particular selected from (S)-1[2′-deoxy-3′,5′-O-(1-phosphono)benzylidene-beta-d-threo-pentofuranosyl]thymine (DPB-T), (+/−)-1-trans-(2-phosphonomethoxycyclopentyl)uracil (PMcP-U), vanillic acid, quercetin, heparin, chondroitin sulphate, etc.); (iii) purine nucleoside phosphorylase (PNPase) inhibitors (more in particular selected from immucillins such as immucillin-H (forodesine, BCX-1777, 1-(9-deazahypoxanthin)-1,4-dideoxy-1,4-imino-D-ribitol), DADMe-immunillin-H and azetidine analogues thereof), such as Ado-phosphorylase (AP) inhibitors.

In another embodiment of the aspects of the present invention, the antibiotic against Mollicutes (b) may be selected from (i) macrolide antibiotics, (more in particular erythromycin, azithromycin or clarithromycin), (ii) tetracyclines (more in particular doxycycline or minocycline) and (iii) fluoroquinolones (more in particular ciprofloxacin or levofloxacin). In another embodiment of the present invention, the antibiotic (b) may be selected from antibiotics active (e.g. with IC₅₀<100 μg/ml) against at least one of Phytoplasma, Ureaplasma, Entomoplasma, Anaeroplasma, Spiroplasma, Mycoplasma mycoides, Mycoplasma pirum, Mycoplasma orale, Mycoplasma arginini, Mycoplasma genitalium, Mycoplasma hominis, Acholeplasma laidlawii, Mycoplasma penetrans, Mycoplasma fermentans, Mycoplasma pneumoniae, Mycoplasma ovipneumoniae, Mycoplasma hiopneumoniae or Mycoplasma hyorhinis.

This invention also relates to a combination of therapeutic agents comprising: (a) a nucleoside-based anti-cancer drug susceptible to inactivation by an enzyme (A), said enzyme (A) being expressed by bacteria (B) which belong to the class of Mollicutes and said enzyme (A) being selected from the group consisting of pyrimidine nucleoside phosphorylases, nucleotidases, purine nucleoside phosphorylases, and deaminases, and (b) a therapeutic agent selected from the group consisting of pyrimidine nucleoside phosphorylase inhibitors, nucleotidase inhibitors, purine nucleoside phosphorylase inhibitors, deaminase inhibitors, and antibiotics against said bacteria (B), for use in the treatment of cancer in a mammal infected with said bacteria (B), provided that said nucleoside-based anti-cancer drug (a) does not require activation by a mammalian homologue of enzyme (A) or in a particular embodiment does not require human thymidine phosphorylase for activation (such as capecitabine, 5-fluoro-5′-deoxyuridine (5′-DFUR) or ftorafur).

This invention also relates to a combination of therapeutic agents comprising: (a) a nucleoside-based anti-cancer drug susceptible to inactivation by an enzyme (A) selected from the group consisting of pyrimidine nucleoside phosphorylases, nucleotidases, purine nucleoside phosphorylases and deaminases, and (b) an antibiotic against bacteria (B) which belong to the class of Mollicutes.

The latter combination is useful in the treatment of cancer in a mammal infected with said bacteria (B).

According to an important aspect of the above combinations of therapeutic agents, the cancer to be treated may be a cancer involving a tumor which does not express said enzyme (A).

Important embodiments of the above combinations of therapeutic agents according to the present invention include one or more of the following features:
combinations of therapeutic agents wherein said nucleoside-based anti-cancer drug (a) is selected from the group consisting of troxacitabine, sapacitabine, 5-fluorouracil, 5-trifluorothymidine, 5-fluoro-dUrd, 6-thioguanine, 6-mercaptopurine, azathioprine, nelarabine, 2′,2′-difluoroguanosine, clofarabine, cladribine, gemcitabine, fludarabine and 5-halogeno-dUrd derivatives;
combinations of therapeutic agents wherein said therapeutic agent (b) is a thymidine phosphorylase inhibitor;
combinations of therapeutic agents wherein said therapeutic agent (b) is 5-chloro-6-(1-[2-imino-pyrrolidinyl]methyl)uracil hydrochloride;
combinations of therapeutic agents wherein said therapeutic agent (b) is an uracil derivative, a solvate or a pharmaceutically acceptable salt thereof other than 5-chloro-6-(1-[2-iminopyrrolidinyl]methyl)uracil hydrochloride, said uracil derivative being represented by the structural formula (I)

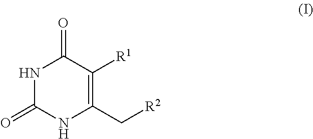

wherein:
R¹ is chloro, bromo, iodo, cyano or C₁₋₄alkyl; and
R² is a 4-8 membered heterocyclic group having 1, 2 or 3 nitrogen atoms, which may be substituted by one or more substituents independently selected from the group consisting of C₁₋₄alkyl, imino, hydroxyl, hydroxymethyl, methanesulfonyloxy, amino and nitro; or R² is an amidinothio group, the nitrogen atoms of which may each be independently substituted by C₁₋₄alkyl; or R² is a guanidino group, the nitrogen atoms of which may each be independently substituted by C₁₋₄alkyl or cyano; or R² is C₁₋₄ alkyl-amidino; or R² is amino, mono-C₁₋₄ alkylamino or di-C₁₋₄alkylamino; or R² is a group with the structural formula—CH₂N(R^{a})R^{b} wherein R^{a} and R^{b} are independently hydrogen or C₁₋₄alkyl or R^{a} and R^{b} may form a pyrrolidine ring together with the nitrogen atom to which they are bonded; or R² is a group with the structural formula —NH—(CH₂)ₘ—Z wherein Z is cyano, amino, mono-C₁₋₄alkylamino or di-C₁₋₄alkylamino, and m is an integer from 0 to 3; or R² is a group with the structural formula NR^{c}(CH₂)ₘ—OH in which R^{c} is hydrogen or C₁₋₄alkyl, and n is an integer from 1 to 4; or R² is a group with the structural formula—X—Y in which X is S or NH, and Y is selected from the group consisting of 2-imidazolin-2-yl, 2-imidazolyl, 1-methylimidazol-2-yl, 1,2,4-triazol-3-yl, 2-pyrimidyl and 2-benzimidazolyl group; or R² is a ureido or thioureido group, the nitrogen atoms of which may each be independently substituted by C₁₋₄alkyl.
combinations of therapeutic agents wherein in said structural formula (I) R₂ is selected from the group consisting of 1-azetidinyl, 1-pyrrolidinyl, 2-pyrrolin-1-yl, 3-pyrrolin-1-yl, 1-pyrrolyl, 1-pyrazolidinyl, 2-pyrazolin-1-yl, 3-pyrazolin-1-yl, 4-pyrazolin-1-yl, 1-pyrazolyl, 1-imidazolidinyl, 2-imidazolin-1-yl, 3-imidazolin-1-yl, 4-imidazolin-1-yl, 1-imidazolyl, 1,2,3-triazol-1-yl, 1,2,4-triazol-1-yl, piperidinyl, 1-piperazyl, morpholino, 1-perhydroazepinyl, 1-perhydroazocinyl, amidino-thio, N-methylamidinothio, N,N′-dimethylamidinothio, 1-guanidino, 1-methylguanidino, 3-methylguanidino, 2,3-dimethylguanidino, 2-cyano-3-methylguanidino, acetoamidino, N-methylamino, N,N-dimethylamino, N-ethylamino, N,N-diethylamino, N-propylamino, N-isopropylamino, N-methylaminomethyl, N,N-dimethylaminomethyl, 1-pyrrolidinylmethyl, N,N-dimethylhydrazino, N-(2-aminoethyl)amino, N-(2-(N,N-dimethyl)amino-ethyl)amino, N-(3-aminopropyl)amino, N-(2-cyanoethyl)amino, N-(2-hydroxyethyl)-N-methylamino, N-(3-hydroxypropyl)amino, N-(4-hydroxy-butyl)amino, 2-imidazolin-2-thio, 2-imidazolin-2-amino, imidazol-2-thio, 1-methylimidazol-2-thio, 1,2,4-triazol-3-thio, pyrimidin-2-thio, benzimidazol-2-thio and 3-methylthioureido;
combinations of therapeutic agents wherein in said structural formula (I) R₁ is bromo, cyano or methyl;
combinations of therapeutic agents wherein said uracil derivative, a solvate or a pharmaceutically acceptable salt thereof as defined in said structural formula (I) is selected from the group consisting of 5-chloro-6-(1-pyrrolidinylmethy)uracil, 5-bromo-6-(1-pyrrolidinylmethyl)uracil, 5-chloro-6-(1-azetidinylmethyl)-uracil, 5-bromo-6-(1-(2-iminopyrrolidinyl)-methyl)uracil hydrochloride, 5-cyano-6-(1-(2-iminopyrrolidinyl)methyl)-uracil, 5-chloro-6-(1-(2-imino-imidazolidinyl)methyl)uracil, 5-bromo-6-(1-(2-iminoimidazolidinyl)-methyl)uracil, 5-chloro-6-(1-imidazolyl-methyl)uracil hydrochloride, 2-(5-chlorouracil-6-ylmethyl)isothiourea hydrochloride, 2-(5-cyanouracil-6-ylmethyl)isothiourea hydrochloride and 5-chloro-6-(1-guanidino)methyl-uracil hydrochloride;
combinations of therapeutic agents wherein said therapeutic agent (b) is selected from the group consisting of pyrimidine nucleoside phosphorylase inhibitors, nucleotidase inhibitors, purine nucleoside phosphorylase inhibitors and deaminase inhibitors, and wherein the molar ratio between said nucleoside-based anti-cancer drug (a) and said therapeutic agent (b) ranges from about 25:1 to 0.01:1, e.g. from about 20:1 to 0.1:1, e.g. from about 20:1 to 4:1;
combinations of therapeutic agents wherein said bacteria (B) are selected from the group consisting of Mycoplasma sp., Acheloplasma sp., Ureaplasma sp., Phytoplasma sp. and Spiroplasma sp.;
combinations of therapeutic agents wherein said antibiotic against bacteria (B) is a Mycoplasma-specific antibiotic;
combinations of therapeutic agents wherein said antibiotic against bacteria (B) is selected from the group consisting of plasmocin, herbicolin A, tetracyclines (e.g. doxycycline or minocycline), (fluoro)quinolones (e.g. ciprofloxacin, enrofloxacin or levofloxacin), macrolides (e.g. azithromycin, erythromycin or clarithromycin) and linkomycin;
combinations of plasmocin with an anti-cancer drug (a) selected from the group consisting of 5-trifluorothymidine, 5-fluorouracil, 5-fluoro-dUrd, 6-thioguanine, 6-mercaptopurine, troxacitabine, sapacitabine, azathioprine, nelarabine, 2′,2′-difluoroguanosine, clofarabine, cladribine, fludarabine, gemcitabine, cytarabine, 5-halogeno-dUrd derivatives;
combinations of therapeutic agents wherein the molar ratio between said nucleoside-based anti-cancer drug (a) and said antibiotic (b) against bacteria (B) ranges from about 10:1 to 0.01:1, e.g. from about 5:1 to 0.1:1, e.g. from about 5:1 to 1:1;
combinations for use in said treatment by consecutive administration, wherein said therapeutic agent (b) is administered prior to said nucleoside-based anticancer drug (a), especially wherein said therapeutic agent (b) is administered from 1 to 5 days, for example 1 to 4 days prior to said nucleoside based anticancer drug (a).

In another aspect the present invention relates to co-cultures of:

(A) an enzyme-negative mammalian tumor cell line, said enzyme being selected from the group consisting of pyrimidine nucleoside phosphorylases, nucleotidases, purine nucleoside phosphorylases and deaminases, and
(B) bacteria belonging to the class of Mollicutes.

Important embodiments of the above co-cultures of the present invention include one or more of the following features:
co-cultures wherein said bacteria (B) are capable of expressing an enzyme selected from the group consisting of pyrimidine nucleoside phosphorylases, nucleotidases, purine nucleoside phosphorylases and deaminases;
co-cultures wherein said tumor cell line (A) is selected from the group consisting of sarcomas, carcinomas, leukemias and lymphomas;
co-cultures wherein said tumor cell line (A) is selected from the group consisting of MCF-7 mammary carcinoma cell line, PC3 prostate cancer cell line, and head and neck squamous carcinoma cell line;
co-cultures wherein said bacteria (B) are selected from the group consisting of Mycoplasma sp., Acheloplasma sp., Ureaplasma sp., Phytoplasma sp. and Spiroplasma sp.;
co-cultures wherein said tumor cell line (A) is a MCF-7 mammary carcinoma cell line and said bacteria (B) is Mycoplasma hyorhinis;
co-cultures for use as a screening tool for an anti-tumor medicament, especially wherein said medicament is a combination comprising (a) a nucleoside-based anti-cancer drug susceptible to inactivation by an enzyme selected from the group consisting of pyrimidine nucleoside phosphorylases, nucleotidases, purine nucleoside phosphorylases and deaminases and (b) a therapeutic agent selected from the group consisting of pyrimidine nucleoside phosphorylase inhibitors, nucleotidase inhibitors, purine nucleoside phosphorylase inhibitors, deaminase inhibitors and antibiotics against bacteria (B) which belong to the class Mollicutes and which express said enzyme.

Nucleoside and nucleotide analogues are widely used as chemotherapeutic agents in the treatment of cancer. Several cancers are reported to be comprise mycoplasmas (i.e. Mycoplasma hyorhinis), which contain a number of nucleoside-metabolizing enzymes. Pyrimidine nucleoside analogues, such as 5-fluoro-2′-deoxyuridine (FdUrd), 5-trifluorothymidine (TFT) and 5-halogenated 2′-deoxyuridines can be degraded by thymidine phosphorylase (TP) to their inactive bases. We found that in Mycoplasma-infected MCF-7 breast carcinoma cells (MCF-7/HYOR) mycoplasma-encoded nucleoside metabolizing enzyme dramatically (20- to 150-fold) reduces the cytostatic activity of the anti-cancer compounds. The reduction in cytostatic activity could be fully restored in the presence of inhibitors of the enzyme. This observation is in agreement with the markedly decreased formation of active metabolite (i.e. FdUMP for FdUrd) or diminished drug incorporation into nucleic acids (i.e. for TFT and 5-bromo-2′-deoxyuridine) in MCF-7/HYOR cells compared with uninfected MCF-7 cells. Antimetabolite formation is fully restored in the presence of the inhibitor.

In contrast, 5-fluoro-5′-deoxyuridine (5′DFUR), an intermediate metabolite of capecitabine, was markedly more cytostatic in MCF-7/HYOR cells than in uninfected cells, due to the activation of this prodrug by the mycoplasma-encoded enzyme.

The present invention therefore provides for the use of a combination therapy for cancer in which a nucleoside- or nucleotide based anti-cancer drug (excluding capecitabine and ftorafur and 5′DFUR anti-cancer therapy) is combined with a mycoplasmal nucleoside or nucleotide-metabolising enzyme inhibitor or an anti-mycoplasma antibiotic.

The present invention clearly shows that mycoplasma infections strongly influence the cytostatic properties of several anti-cancer agents such as fluoropyrimidine analogues. The results reveal that Mycoplasma-encoded enzymes significantly decrease the accumulation of cytostatic nucleoside metabolites into the tumor cells and markedly down-modulates the cytostatic activity of these compounds. Administration of a specific mycoplasma enzyme inhibitor and/or mycoplasma antibiotic with the anti-cancer nucleoside or nucleotide analogues can fully restore the cytostatic activity in the mycoplasma-infected cell cultures (Bronckaers et al., 2008; 76:188-197; Liekens et al. 2009; Lancet Oncol. in press).

The present invention also relates to a product comprising at least (a) a nucleoside- or nucleotide-based anti-cancer drug and (b) an agent selected from an inhibitor of a mycoplasma nucleoside or nucleotide metabolising enzyme and/or a mycoplasma antibiotic.

The present invention also concerns a product comprising at least (a) a nucleoside- or nucleotide-based anti-cancer drug and (b) an agent selected from (i) an inhibitor of a mycoplasma nucleoside or nucleotide metabolising protein and (ii) a mycoplasma antibiotic.

The present invention also relates to a pharmaceutical composition comprising a pharmaceutically acceptable carrier and as active ingredients the product described above. The pharmaceutical composition can be presented as a combined preparation for simultaneous, separate or sequential use for the treatment or prevention of cancer (including tumor formation, growth and metastasis). In an embodiment, the pharmaceutical composition comprises at least (a) a nucleoside- or nucleotide-based anti-cancer drug and (b) an agent selected from an inhibitor of a mycoplasma nucleoside metabolising enzyme and/or a mycoplasma antibiotic, as a combined preparation for simultaneous, separate or sequential use in the treatment of cancer, provided that the nucleoside- or nucleotide-based anti-cancer drug is not an anti-cancer drug that requires human cellular enzymes to be activated such as capecitabine or ftorafur, and provided that the nucleoside- or nucleotide-based anti-cancer drug is not TFT when the agent selected from an inhibitor of a mycoplasma-nucleoside metabolising enzyme and/or a mycoplasma antibiotic is TPI. Preferably, the pharmaceutical composition comprises (a) a nucleoside- or nucleotide-based anti-cancer drug and (b) a mycoplasma antibiotic.

The present invention also relates to a method for the prevention or treatment of cancer in an animal (more particularly a mammal or a human), comprising providing and administering to said animal an effective amount of said product of said pharmaceutical composition.

The present invention also relates to a method for the prevention or treatment of cancer in an animal (more particularly a mammal or a human), wherein an effective amount of a pharmaceutical composition, said pharmaceutical composition comprising a pharmaceutically acceptable carrier and as active ingredients (a) a nucleoside- or nucleotide-based anti-cancer drug and (b) an agent selected from an inhibitor of a mycoplasma nucleoside or nucleotide metabolising enzyme and/or a mycoplasma antibiotic, is provided and/or administered to said animal. In a particular embodiment, the (a) nucleoside- or nucleotide-based anti-cancer drug and (b) inhibitor of a mycoplasma nucleoside or nucleotide metabolising enzyme and/or mycoplasma antibiotic are administered simultaneous to the animal. In another particular embodiment, the method for the prevention or treatment of cancer in an animal (more particularly a mammal or a human), comprises providing and administering to said animal an effective amount of a pharmaceutical composition, said pharmaceutical composition comprising a pharmaceutically acceptable carrier and as active ingredients (a) a nucleoside- or nucleotide-based anti-cancer drug and (b) a mycoplasma antibiotic, in which the mycoplasma antibiotic is administered at least 1 day, preferably between 1 and 5 days, such as 3 days, before or simultaneous to the administration of the a nucleoside- or nucleotide-based anti-cancer drug.

Since the present invention only works for anti-cancer agents which are degraded by mycoplasma metabolising enzymes, and not for anti-cancer agents which are not degraded or even are activated by (mycoplasma or human) metabolising enzymes, the product and the pharmaceutical compositions of the present invention does not comprise nucleoside- or nucleotide-based anti-cancer drug that require (e.g. human) nucleotide or nucleotide metabolising enzymes to be activated, such as for capecitabine, 5-fluoro-5′-deoxyuridine (5′DFUR) or ftorafur. This is especially in the situation that the inhibitor of a mycoplasma nucleoside or nucleotide metabolising protein is also active as inhibitor of the human or mammalian nucleotide or nucleotide metabolising enzyme homolog.

The present invention also relates to the use of (a) a nucleoside- or nucleotide-based anti-cancer drug and (b) an agent selected from an inhibitor of a mycoplasma nucleoside or nucleotide metabolising enzyme and/or a mycoplasma antibiotic, for the preparation of a product or pharmaceutical composition of the invention or for the manufacture of a medicament for the prevention or treatment of cancer.

Since also the combination of TFT and a TP inhibitor is already being applied as combination therapy, the product and pharmaceutical composition of the present invention also does not comprise the combination of the nucleoside- or nucleotide-based anti-cancer drug TFT with an inhibitor of a mycoplasma nucleoside metabolising protein, more in particular TPi. The combination of TFT with TPi is based on the fact that TFT is highly metabolised by human metabolising enzymes. No mention is made of the fact that mycoplasma-metabolising enzymes are involved in the deactivation of TFT or of other anti-cancer agents.

The nucleoside- or nucleotide-based anti-cancer drugs to be used in the invention can be selected from (i) pyrimidine-based anti-cancer drugs (meaning drugs which comprise a pyrimidine structure, such as 5-fluorouracil) and (ii) purine based anti-cancer drugs (meaning drugs which comprise a purine structure, such as azathioprine or adenine).

The purine based anti-cancer drugs are selected from adenine derivatives (comprising a substituted or unsubstituted 6-amino-purine structure) and guanine derivatives (comprising a substituted or unsubstituted 2-amino-purin-6-one structure). Examples of the purine based anti-cancer drugs are selected from mercaptopurine (6 MP), thioguanine (6TG), azathioprine, fludarabine, cladribine and clofarabine, araG and dFdG.

The pyrimidine based anti-cancer drugs can be selected from thymine derivatives (comprising a substituted or unsubstituted 5-methylpyrimidine-2,4-dione structure), cytosine derivatives (comprising a substituted or unsubstituted 4-amino-pyrimidine-2-one structure) and uracil derivatives (comprising a substituted or unsubstituted pyrimidine-2,4-dione structure). Examples of the pyrimidine based anti-cancer drugs are selected from cytarabine (araC), gemcitabine (dFdC), fluorouracil (FU), 5-fluoro-2′-deoxyuridine (5FdUrd), trifluorothymidine (TFT), capecitabine, 5′DFUR and ftorafur.

The following combinations can be used according to the present invention:
a TP inhibitor such as 5-chloro-6-(1 [2-iminopyrrolidinyl]methyl)uracil hydrochloride (hereinafter TPi) with 5-halogeno-dUrd, or araC or gemcitabine or cladribine or clofarabine;
an adenosine phosphorylase inhibitor or PNPase inhibitor such as immucillin H and cladribine or clofarabine.

The inhibitors of a mycoplasma-nucleoside metabolising enzymes can be selected from any known inhibitor or these proteins such as (i) pyrimidine phosphorylase inhibitors such as thymidine phosphorylase (TP) inhibitors (more in particular selected from TPi) and uridine phosphorylase (UP) inhibitors, (ii) nucleotidase inhibitors (more in particular selected from (S)-1[2′-deoxy-3′,5′-O-(1-phosphono)benzylidene-beta-d-threo-pentofuranosyl]thymine (DPB-T), (+/−)-1-trans-(2-phosphonomethoxycyclopentyl)uracil (PMcP-U), vanillic acid, quercetin, heparin, chondroitin sulphate,), such as nucleotidase inhibitors and (iii) purine nucleoside phosphorylase (PNPase) inhibitors (more in particular selected from immucillins such as immucillin-H (forodesine, BCX-1777, 1-(9-deazahypoxanthin)-1,4-dideoxy-1,4-imino-D-ribitol), DADMe-immucillin-H and azetidine analogs thereof), such as Ado-phosphorylase inhibitors.

The mycoplasma antibiotics can then again be selected from anti-bacterial agents having an inhibitory or lethal activity on at least one mycoplasma species (such as Mycoplasma mycoides, M. pirum, M. penetrans, M. fermentans, M. pneumoniae and M. hyorhinis). Examples are (i) macrolide antibiotics, more in particular the azalide macrolide antibiotics (more in particular erythromycin, azithromycin and clarithromycin), (ii) tetracyclines (more in particular doxycycline and minocycline) and (iii) the fluoroquinolones (more in particular ciprofloxacin and levofloxacin). Since mycoplasmas do not comprise a cell wall, the mycoplasma antibiotics are not selected from antibiotics of which the mechanism of action for the anti-bacterial activity involves the cell wall.

Some examples of anti-cancer agent metabolisation known in the prior art and the effect of the present invention are described in detail.

The fluoropyrimidine 5-fluorouracil (5FU) is successfully used against a variety of solid tumors, including breast, oesophageal and colon carcinoma. 5FU elicits its antitumor activity primarily by inhibiting thymidylate synthase (TS), a rate-limiting enzyme in DNA synthesis. This requires conversion of 5FU to 5-fluoro-2′-deoxyuridine 5′-monophosphate (FdUMP), which inhibits TS. However, the clinical efficacy of 5FU is limited by its rapid degradation [by dihydropyrimidine dehydrogenase (DPD)] and poor oral bioavailability. Therefore, efforts have been made to develop oral 5FU-prodrugs. Doxifluridine (5′-deoxy-5-fluorouridine, 5′DFUR) is a prodrug of 5FU that requires thymidine phosphorylase (TP) for its one-step conversion to 5FU. However, 5′DFUR therapy resulted in dose-limiting gastrointestinal toxicity. Capecitabine (N-4-pentyloxycarbonyl-5′-deoxy-5-fluorocytidine, Xeloda®) was designed to circumvent this toxicity by more selectively delivering 5FU to the tumor. Capecitabine is converted to 5FU in three distinct steps. It is first converted to 5′-deoxy-5-fluorocytidine by carboxylesterase in the liver, then to 5′-deoxy-5-fluorouridine (5′-DFUR) by cytidine deaminase and finally to 5FU by TP. Currently, capecitabine is being used for the treatment of metastatic breast and colorectal cancers.

TP is not only a key enzyme in the pyrimidine nucleoside salvage pathway; it is also identical to platelet-derived endothelial cell growth factor (PD-ECGF), an angiogenic factor with anti-apoptotic properties. Increased TP levels are found in several solid tumors and are correlated with high neovascularisation, increased metastasis and poor prognosis. Nevertheless, high TP levels improve the effectiveness of 5FU prodrug-based chemotherapy.

In spite of good therapeutic results, a large number of patients eventually acquire resistance against 5FU-based chemotherapy. The fluoropyrimidine nucleoside 5-trifluorothymidine (TFT) has been shown to bypass this resistance. The mechanism of cytostatic action of TFT is based on inhibition of TS as its monophosphate and incorporation of the drug into the DNA after conversion to its triphosphate metabolite. However, TFT is rapidly inactivated by TP, which converts TFT to its inactive base. Therefore, a new drug formulation containing TFT and a potent inhibitor of mammalian TP [5-chloro-6-(1-[2-iminopyrrolidinyl]methyl)uracil hydrochloride (TPi)], designated TAS-102, has been developed. At present, TAS-102 is being evaluated in phase I clinical trials for the treatment of various solid tumors. Thus, TP has an ambiguous role in fluoropyrimidine-based chemotherapy. It may enhance the anti-tumoral properties of 5FU prodrugs such as capecitabine on the one hand, but it may inactivate pyrimidine 2′-deoxyuridine derivatives, such as TFT, on the other hand.

TP activity is not only upregulated in tumors, it is also expressed by several mycoplasma species, such as Mycoplasma mycoides and M. pirum. Mycoplasmas are the smallest self-replicating bacteria and are important human pathogens. They can cause severe respiratory and urogenital diseases. Most mycoplasma infections, however, remain unidentified, because many people seem to be chronically infected with mycoplasmas without apparent clinical symptoms. A possible association between mycoplasmas and leukaemia has already been suggested in the 1960's (Haflick L et al., Nature 1965; 205:713-4; Cimolai N. et al., Can J Microbiol 2001; 47:691-7). More recently, mycoplasmas were detected in tissues of ovarian and cervical cancer, by using sensitive PCR-ELISAs (Kidder M et al., Gynecol Oncol 1998; 71:254-7; Chan P J et al., Gynecol Oncol 1996; 63:258-60). In addition, Mycoplasma penetrans was found to be associated with Kaposi's sarcoma. Immunohistological analysis of carcinoma tissues, demonstrated a significant correlation between the presence of M. hyorhinis and gastric and colon cancer. Whether the mycoplasmas cause the cancers or their presence is a consequence of the cancer has never been established.

Thus, a number of studies have highlighted the presence of mycoplasmas in cancer, but no clinically relevant causal link between mycoplasmas and cancer has been proven. Chronic mycoplasma infections with M. penetrans and M. fermentans induced chromosomal instability in C3H murine embryonic cells, prevented apoptosis and caused malignant transformation in 32D haematopoietic cells. When injected into nude mice, these transformed 32D cells quickly developed tumors, while the control cells did not. Infection with some strains of M. fermentans promoted immortalization of human peripheral blood mononuclear cells in culture. Mycoplasma hyorhinis was found to express p37, a protein that increases the invasiveness of prostate and melanoma cell lines in vitro. This protein also altered gene expression, growth and migratory potential of the prostate cancer cell lines PC-3 and DU145. Recent data indicate that p37 promotes cancer cell invasiveness and metastasis by activation of MMP-2 and by phosphorylation of the epidermal growth factor receptor.

Until now, no anti-cancer treatment has been established with anti-mycoplasma agents or agents targeting mycoplasma nucleoside or nucleotide metabolising enzymes.

Our research now revealed that mycoplasma species such as M. hyorhinis play a thus far underestimated detrimental role in compromising the cytostatic activity of certain nucleoside drugs such as FdUrd and TFT, but also in improving the cytostatic activity of TP-dependent prodrugs of 5FU such as capecitabine. In addition, we showed that a specific human TP inhibitor (i.e. TPi) is able to efficiently inhibit this mycoplasma-encoded enzyme, fully restore the impaired active metabolite formation of the pyrimidine nucleoside analogues and concomitantly the drugs' cytostatic potential. TAS-102, a combination of TFT and TPi is currently subject of phase I clinical trials for the treatment of various solid tumors. This therapy seems to enhance the anti-tumor properties and to decrease the toxicity of TFT. An additional advantage of this combination therapy would be that it can also inhibit TP of mycoplasmas that may be associated with the treated cancer, thus preventing a premature breakdown of TFT in human plasma and/or tumor tissue of mycoplasma-infected cancer patients.

Mycoplasmal contaminations are a recurrent problem in the use of cell cultures. Studies pointed out that 10 to 80% of cell cultures are infected by mycoplasmas. M. hyorhinis but also M. orale, M. arginini, M. fermentans and Acholeplasma laidlawii are commonly found in such cell cultures. The sources of mycoplasma contaminations in cell cultures are usually culture reagents (fetal calf serum), cross-contamination from infected cell cultures and infections that originate from the laboratory staff [51]. Numerous reports have stated that mycoplasma infections of cell cultures can lead to unreliable experimental results [37, 51]. For example, they can alter cell metabolism, protein synthesis, RNA and DNA synthesis, cell membrane composition and cell morphology, and they can trigger cell death [51]. Our data demonstrate that mycoplasma infections may also interfere with the eventual cytostatic activity of a variety of nucleoside analogues. Therefore, laboratories that investigate antitumoral properties of nucleoside analogue drugs should remove mycoplasmas from their cell cultures and establish an effective routine mycoplasma screening program.

Our findings have high relevance for cancer treatment with nucleoside anti-cancer drugs such as FdUrd and TFT. M. hyorhinis is frequently found in tissues of gastric, colon, oesophageal, lung and breast cancer, but not in analogous non-tumorigenic tissue. Our data reveal that the presence of this mycoplasma species markedly compromises the cytostatic efficacy of several nucleoside-based chemotherapeutic agents. We show that nucleoside-based anti-cancer chemotherapy should be combined with a mycoplasma enzyme inhibitor and/or a specific antibiotic directed against mycoplasmas to prevent premature inactivation of the drug in the plasma and at the site of the tumor.

### Examples

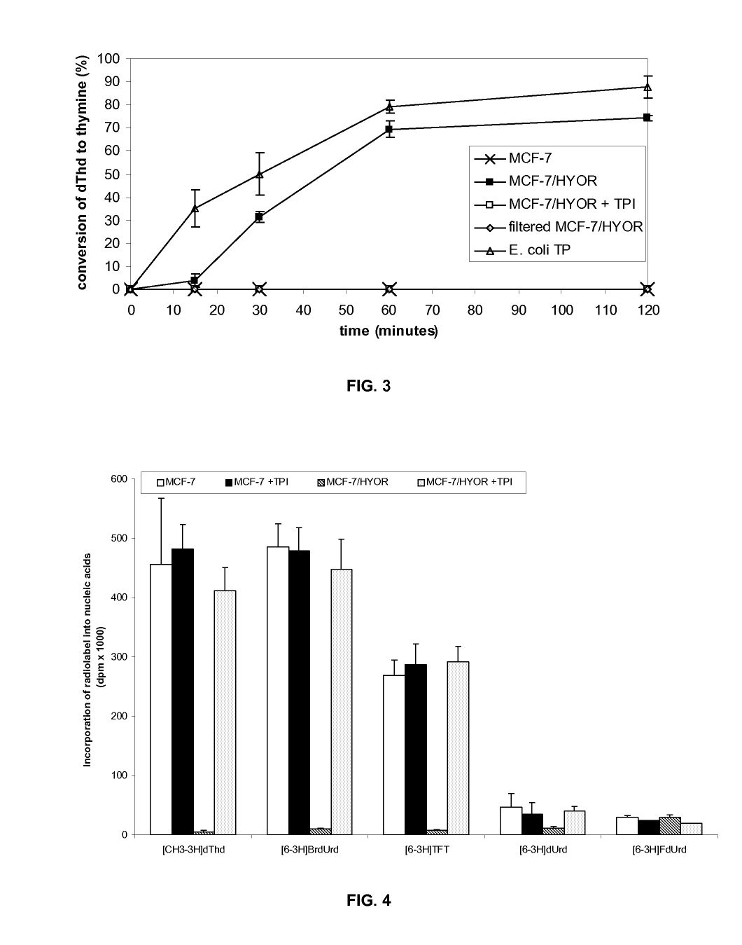

The present invention has been established by focusing in first instance on TP. TP is an enzyme of the pyrimidine nucleoside salvage pathway that catalyzes the reversible conversion of thymidine and phosphate into thymine and 2-deoxy-D-ribose-1-phosphate. Previously, TP activity has been detected in the mycoplasma species Mycoplasma pirum and Mycoplasma mycoides. Others have reported that [³H]-thymidine incorporation into DNA was impaired in cell cultures contaminated with mycoplasmas, suggesting an enzymatic cleavage of thymidine by TP activity originating from mycoplasmas. In the present study, we report that also M. hyorhinis contains TP activity. Moreover, we show that the TP encoded by this mycoplasma species not only catalyzes the conversion of thymidine to thymine, it also efficiently recognizes FdUrd, TFT and 5′DFUR, which are known substrates of E. coli and mammalian TPs. Although the enzymatic activity of TP is reversible, the equilibrium of this reaction is towards the nucleobase and not towards the pyrimidine nucleoside. Within 60 minutes almost all thymidine is degraded into thymine (FIG. 3). These results are in line with the previously reported pronounced phosphorolysis of thymidine by E. coli TP or TP extracted from human platelets. Infection of TP-negative MCF-7 cells by M. hyorhinis did not induce the expression of human TP as was demonstrated by Western blot analysis on cell lysates of MCF-7/HYOR cells (FIG. 2). Thus, the effects observed in the M. hyorhinis-infected MCF-7 cell cultures were due to the expression of mycoplasma-specific TP and not to upregulated or induced human TP. TP produced by M. hyorhinis significantly decreased the sensitivity of MCF-7 cells to the antiproliferative activity of FdUrd, TFT and other 5-halogen-substituted dUrd analogues. The reduced antiproliferative activities of these cytostatic compounds in MCF-7/HYOR cell cultures could be fully restored by adding TPi, a well-known human TP inhibitor, but also by adding the anti-mycoplasmal antibiotic plasmocin (25 μg/ml) to the cells three days prior to addition of the drugs. Plasmocin efficiently inhibits DNA replication and protein synthesis of mycoplasma (plasmocin.com). These observations again demonstrate that mycoplasma-encoded enzyme(s) (i.e. TP) may markedly compromise the cytostatic action of the nucleoside analogues. Thus, M. hyorhinis TP efficiently converts FdUrd, TFT and other 5-halogen-substituted dUrd, to their respective free pyrimidine bases. However, previously it has been reported that transfection of MCF-7 and KB cells with human TP does not significantly alter the cytotoxic activity of FdUrd. The markedly reduced sensitivity of MCF-7/HYOR cell cultures to the cytostatic activity of FdUrd (and TFT) may therefore suggest that M. hyorhinis TP has a better substrate affinity for FdUrd and/or a higher catalytic activity than human TP in the transduced MCF-7/TP cells. Alternatively, our data may also point to a much faster inactivation of the drugs by M. hyorhinis TP in the extracellular medium than uptake and activation by the anabolic cellular thymidine kinase in MCF-7 cells. Further studies are needed to clarify the issues.

The markedly decreased incorporation of dThd, TFT and BrdUrd in MCF-7/HYOR nucleic acids and the decreased formation of FdUrd 5′-monophosphate in MCF-7/HYOR cells are in line with our findings that M. hyorhinis encoded-TP prevents the cytostatic activity of these drugs (FIG. 4, Table 4). Thus, mycoplasma-infected tumor tissue, a phenomenon seen in a variety of tumors, may render pyrimidine nucleoside-based anticancer therapy markedly less efficient. Instead, the TP-dependent fluoropyrimidine prodrug capecitabine is efficiently activated by mycoplasmal TP in TP-negative MCF-7/HYOR tumor cells (Table 2). Indeed, 5′DFUR, which is an intermediate metabolite of capecitabine, was markedly more cytostatic in mycoplasma-infected MCF-7/HYOR cells. The increased cytostatic activity of 5′DFUR in MCF-7/HYOR cell cultures was efficiently annihilated by TPi. Transfection of the human TP gene into cancer cell lines such as MCF-7, KB, HT-29 and PC-9 was also shown to increase the sensitivity to 5′DFUR in comparison to the parental cell lines, providing direct evidence for the role of TP in 5′DFUR sensitivity. Thus, successful outcome of capecitabine treatment highly depends on the TP activity of the tumors. Therefore, clinical therapies that upregulate TP expression, such as taxanes and X-ray irradiation, have been shown to improve the effectiveness of capecitabine. Since mycoplasmas such as M. hyorhinis abundantly express TP, capecitabine sensitivity may be further increased in tumor tissue containing mycoplasmas.

The inventors have also shown in the present invention that M. hyorhinis infection significantly reduces the anti-proliferative effect of the cytidine analogue gemcitabine (2′,2′-difluorodeoxycytidine) by 10- to 70-fold, depending on the nature of the tumor cell line (Liekens et al., Lancet Oncology, in press, 2009). For example, M. hyorhinis infection of human osteosarcoma (OST.TK⁻) and breast carcinoma (MDA-MB-231 and MCF-7) cell lines respectively resulted in a 70-, 40- and 10-fold reduction in the cytostatic activity of gemcitabine. By means of flow cytometry it was shown that gemcitabine causes MCF-7 cell cycle arrest in the S-phase at a concentration of 0.2 μM. In contrast, a 25-fold higher concentration of gemcitabine was needed to cause a similar effect in MCF-7/HYOR cells. Using radiolabeled gemcitabine, the incorporation of its active metabolite was compared in MCF-7 and MCF-7/HYOR DNA. A 15- to 60-fold reduction of gemcitabine-triphosphate incorporation in MCF-7/HYOR DNA was observed. HPLC analysis revealed that the presence of mycoplasmas in the tumor cell cultures inhibits the metabolism of gemcitabine, ultimately resulting in a markedly decreased pool of its active triphosphate metabolite. The cytostatic activity of gemcitabine in different human tumor cell lines was shown to be drastically inhibited upon mycoplasma infection. The present inventors show that co-administration of a mycoplasma-specific antibiotic or inhibitor of mycoplasma-enzymes significantly enhance the efficiency of cancer chemotherapy with cytosine analogues, such as gemcitabine.

### Example 1

### Materials and Methods for In Vitro and Cellular Experiments

### Reagents and Materials

TPi, 5-chloro-6-(1 [2-iminopyrrolidinyl]methyl)uracil hydrochloride, a potent inhibitor of TP, is described in literature (Fukushima M. et al., Biochem Pharmacol 2000; 59:1227-36). 5-Fluoro-5′-deoxyuridine (5′DFUR), 5-trifluorothymidine (TFT), thymidine (dThd), 5-fluoro-2′ deoxyuridine (FdUrd), 5-chloro-2′-deoxyuridine (CIdUrd), 5-bromo-2′-deoxyuridine (BrdUrd), 5-iodo-2′-deoxyuridine (IdUrd), and 5-fluorouracil (5FU) were purchased from Sigma (St-Louis, Mo.). Gemcitabine (dFdC) and cladribine were obtained from Prof. McGuigan (Cardiff, UK). [CH₃—³H]-Thymine, [6-³H]-TFT, [2-¹⁴C]-TF-thymine, [6-³H]-BrdUrd, [6-³H]-FdUrd, [6-³H]-dUrd, [5-³H]-uracil, [6-³H]-5FU and [5-³H]-dFdC were obtained from Moravek Biochemicals (Brea, Calif.) and [CH₃—³H]-dThd from MP Biomedicals (Solon, Ohio). Plasmocin was purchased from Invivogen (San Diego, Calif.). The antibody against β-actin was obtained from Sigma, the polycolonal antibody against TP (clone G-19) from Santa Cruz Biotechnology (Santa Cruz, Calif.)

### Cell Culture

TP-negative MCF-7 breast carcinoma cells were kindly provided by Prof. G. J. Peters (Amsterdam, The Netherlands) (Lopez L R et al., Eur J Cancer 1994; 30A:1545-9). The cells were maintained in Dulbecco's modified Eagle's medium (DMEM) (Invitrogen, Carlsbad, Calif.) supplemented with 10% foetal bovine serum (FBS) (Harlan, Sera-Lab Ltd, Loughborough, UK) and 10 mM Hepes (Invitrogen). Cells were grown at 37° C. in a humidified incubator with a gas phase of 5% CO₂. MCF-7 cells overexpressing human TP were obtained by transfection of MCF-7 cells with the TP/PD-ECGF full-length cDNA expression vector that was kindly provided by Prof. S. Akiyama (Haraguchi M. et al., Cancer Res 1993; 53:5680-2).

Culture of M. hyorhinis

Mycoplasma hyorhinis (ATCC 17981) was obtained from the American Type Culture Collection (ATCC, Manassas, Va.). The freeze-dried bacteria were reconstituted by adding 1 ml of DMEM. MCF-7 cells were seeded at 20,000 cells/cm² in DMEM containing 10% FBS (mycoplasma-screened). Two days later, the MCF-7 cell cultures were infected with M. hyorhinis by adding 500 μl of the freshly reconstituted mycoplasmas. The co-culture of MCF-7 cells and M. hyorhinis was maintained under the same conditions as the uninfected MCF-7 cells.

Identification of M. hyorhinis by PCR

To confirm the infection of MCF-7 cells by M. hyorhinis, a species-specific PCR for M. hyorhinis was performed as described by Kong et al. (Kong F. et al., Appl Environ Microbiol 2001; 67:3195-200). All PCR reactions were performed using Taq Polymerase (Sphaero Q, Leiden, The Netherlands). The primers used for the PCR were HYR+(5′ catgatgagtaatagaaaggagcttcacagcttc-3′) and UNI-(5′-ccagggtatctaatcctgtttgctccc-3′), which produce a PCR-fragment of 616 bp long (Haraguchi M. et al., Cancer Res 1993; 53:5680-2). PCR amplification consisted of 40 cycles of denaturation at 96° C. for 1 s, annealing at 68° C. for 1 s and extension at 74° C. for 10 s.

Staining of DNA with Hoechst 33342

10,000 cells/cm² (MCF-7 and MCF-7/HYOR) were seeded in 8-well chamber slides (Nunc, Roskilde, Denmark). After 24 hours, 10 μM TPi was added and the cells were incubated for an additional 72 hours. Next, the cells were fixed with Carnoy's fixative (1 part glacial acetic acid to 3 parts absolute methanol) for 10 minutes, air-dried and exposed to the DNA-binding dye Hoechst 33342 (Sigma) at a concentration of 0.5 μg/ml for 15 min at room temperature. Next, the cells were washed twice with de-ionised water and covered with mounting medium (‘glycergel’, Dako, Glostrup, Denmark) and a cover slip. Fluorescence was visualised with a Leica TCS SP5 confocal microscope (Leica, Wetzlar, Germany).

### Western Blot Assay

MCF-7 and MCF-7/HYOR cells were seeded at 8,000 cells/cm². Forty-eight hours later, the cells were washed with ice-cold phosphate-buffered saline (PBS) and lysed as described previously (Liekens S. et al., Mol Pharmacol 1999; 56:204-13). Lysates were cleared by centrifugation, and the protein concentration of the supernatants was determined. One ml of the culture medium was centrifuged at 1,200 rpm for 5 minutes. The supernatant was sonicated and concentrated 10 times by using a vivaspin concentrator with a cut-off size of 5,000 Da (Sartorius AG, Goettingen, Germany). SDS-polyacrylamide gel electrophoresis of 40 μg of the cell lysates and 20 μl of the concentrated medium was performed after which the proteins were transferred to a Hybond-P polyvinylidene difluoride membrane (GE Healthcare, Little Chalfont, Buckinghamshire, UK). The membranes were incubated for 1 h at room temperature in blocking buffer (5% nonfat dry milk in PBS containing 0.1% Tween 20) and subsequently for 1 h in blocking buffer with primary antibodies raised against β-actin (1/5000) or TP (1/1000). After washing, the membranes were incubated with the corresponding horseradish peroxidase-conjugated secondary antibody (anti-mouse, 1/2000; Dako) in blocking buffer for 25 min at room temperature. Next, the membranes were washed extensively. Immunoreactive proteins were detected by chemiluminescence (ECLplus; GE Healthcare). As a positive control a cell lysate from MCF-7 cells transfected with human TP gene (MCF-7/TP) was loaded on the gel.

### Enzyme Activity Assays

The TP activity of M. hyorhinis and the conversion of dThd, FdUrd, 5′DFUR and TFT to thymine, 5FU, 5FU or TF-thymine respectively were measured by high-pressure liquid chromatography (HPLC) analysis. MCF-7 and MCF-7/HYOR cells were seeded at a density of 20,000 cells/cm² in DMEM with 10% FBS. Four days later, the medium was collected and cleared by centrifugation at 1,400 rpm. For some experiments, the medium of MCF-7/HYOR cells was filtered using a 0.1 μm micro filter (Acrodisc syringe filter, PALL Corporation, East Hills, N.Y.) to remove the mycoplasmas from the medium. 600 μl of the medium was incubated with 200 μM of substrate (dThd, 5′DFUR, TFT or FdUrd) at 37° C. in the presence or absence of 10 μM TPi. At different time points (i.e. 0, 15, 30, 60, 120 minutes and 16 hours), 100 μl aliquots were withdrawn, transferred to Eppendorf tubes and heated at 95° C. for 3 min. Next, the samples were rapidly cooled on ice, exposed for 20 min to 200 μl ice-cold methanol and cleared by centrifugation at 15,000 rpm for 15 minutes. As a positive control, an enzyme activity assay with E. coli TP (Sigma) was performed. For this reaction, 0.025 U of TP were incubated with 200 μM of substrate in TP-buffer (10 mM Tris-HCl, pH 7.6, 1 mM EDTA, 2 mM potassium phosphate and 150 mM NaCl) in a total volume of 600 μl. Aliquots of 100 μl were withdrawn from the reaction mixture at several time points and treated as described above. The nucleosides were separated from their nucleobases on a reversed-phase RP-8 column (Merck, Darmstadt, Germany) and quantified by HPLC analysis (Aliance 2690, Waters, Milford, Mass.). The separation was performed by a linear gradient from 100% buffer B (50 mM NaH₂PO₄ and 5 mM heptane sulfonic acid, pH 3.2), to 20% buffer B and 80% acetonitrile. Retention times of thymine and thymidine were respectively 5.1 and 10.8 minutes. UV-based detection of all nucleosides was performed at 267 nm.

### Tumor Cell Proliferation Assays

MCF-7 and MCF-7/HYOR cells were seeded in 48-well plates at 10,000 cells/cm². After 24 h, different concentrations (e.g. 250 μM, 50 μM, 10 μM, 2 μM, 0.4 μM and 0.08 μM) in order to determine the IC50s. Values are presented as means±S.E.M. of at least three independent experiments of the test compounds (5FU, 5′DFUR, CIdUrd, BrdUrd, IdUrd and TFT) with or without 10 μM TPi were added. The cells were incubated for another 4 days, trypsinized and counted by a Coulter counter (Analis, Suarlée, Belgium). In some experiments, the antibiotic plasmocin was added one or three days before addition of the test compounds.

### Nucleotide Incorporation Assay

MCF-7 and MCF-7/HYOR cells were seeded at 10,000 cells/cm. After 48 hours, cells were treated with 1 μCi of ³H-labeled nucleoside with or without 10 μM TPi. 16 h later, the medium was removed and the cells were washed twice with PBS. Next, the cells were trypsinized, transferred to Eppendorf tubes and centrifuged for 10 minutes at 1,400 rpm. The pellet was resuspended in 1 ml absolute ice-cold methanol and kept on ice for 20 minutes. After centrifugation for 20 minutes at 13,000 rpm the pellet was washed twice with methanol, resuspended in methanol and transferred to scintillation vials containing 9 ml of Ready safe liquid scintillation reagent (‘Hisafe 3’, Perkin Elmer, Waltham, Mass.). The radioactivity was measured by a Liquid scintillation analyzer (2300 TR, Packard, Canberra, Australia).

### Nucleoside Metabolism Experiments

MCF-7 and MCF-7/HYOR cells were seeded and treated with 1 μCi of nucleoside with and without TPi as described above. 16 h later, medium was collected and the cells were washed twice with PBS. Next, the cells were incubated in 0.5 ml absolute ice-cold methanol and kept on ice for 20 minutes. After centrifugation for 20 minutes at 13,000 rpm, the supernatant was subjected to HPLC analysis. The nucleobases, nucleosides and nucleotides in the supernatant were separated by a Partisphere 10 SAX anion exchange column (Whatmann International Ltd., Maidstone, England) as described earlier (Balzarini J. et al., AIDS 2002; 16:2159-63), while the nucleobases and nucleosides present in the collected medium were separated using an RP-8 column. The amount of compound incorporated into nucleic acids was measured as described above.

### Example 2

### Identification of M. Hyorhinis Infection in MCF-7/HYOR Cell Cultures

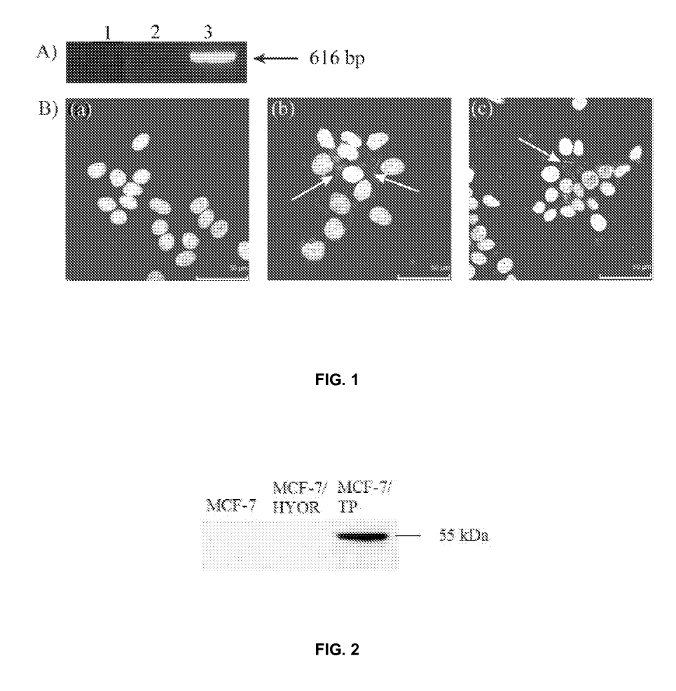

Productive infection of MCF-7 cells with M. hyorhinis was confirmed by a species-specific PCR, which detected a PCR-band of 616 bp in the MCF-7/HYOR cell extracts (FIG. 1A). No PCR-bands were found in the uninfected MCF-7 cell extract or in the non-template control. Infection of MCF-7 cells with M. hyorhinis was also evaluated by staining the cellular and bacterial DNA with the Hoechst 33342 dye (FIG. 1B). Nucleic acid-rich particles can be visualized in the cytosol of the MCF-7/HYOR cells and MCF-7/HYOR cells that were treated for 3 days with TPi (10 μM) indicating that TPi is not inhibitory to the growth of M. hyorhinis in MCF-7 cell cultures.

### Example 3

### Detection of Human TP in MCF-7 and MCF-7/HYOR Cell Extracts and Cell Culture Medium

Western blot analysis using a polyclonal antibody against human TP did not detect the protein in extracts of MCF-7 and MCF-7/HYOR cells (FIG. 2). However, human TP could be abundantly detected in extracts from MCF-7 cells that were transfected with the human TP gene. This confirms that MCF-7 cells do not express human TP and indicates that M. hyorhinis infection does not induce the expression of human TP in MCF-7 cells. Also, human TP was not detected in the medium of uninfected MCF-7 and M. hyorhinis-infected MCF-7/HYOR cells (data not shown). The polyclonal antibody used in this assay, did not show any cross-reactivity with the mycoplasmal TP present in the culture medium of MCF-7/HYOR cells.

### Example 4

### TP Enzyme Activity Assays in the Supernatant of MCF-7/HYOR Cell Cultures

The TP enzyme activity and time-course of the enzymatic reaction were determined in the medium of 4-day-old MCF-7/HYOR cell cultures (Table 1, FIG. 3). Seventy-one % of dThd (200 μM) was converted into thymine within 2 hours. All dThd had disappeared from the reaction mixture after 16 hours. The pyrimidine nucleoside analogues FdUrd, 5′DFUR and TFT were also converted to their respective pyrimidine bases, although to a lesser extent than the natural substrate dThd (Table 1). In the MCF-7/HYOR culture medium, the conversion of all compounds (200 μM dThd, TFT, FdUrd and 5′DFUR) to their respective free bases could be completely inhibited in the presence of 10 μM TPi (a potent inhibitor of human and E. coli TP). In contrast, no conversion of dThd, TFT, FdUrd or 5′DFUR was observed in the medium of uninfected MCF-7 cells, even after 24 hours of incubation (data not shown). Interestingly, no TP activity was found in the filtered (0.1 μm) supernatant of MCF-7/HYOR cell cultures. Thus, by removing the mycoplasmas from the medium, the TP activity in the cell culture medium is lost, indicating that the measured TP activity is bacteria-associated and not extracellularly secreted by the mycoplasmas.

The time-course curve of the TP-activity shows an initial lag-phase (FIG. 3). This may indicate that dThd first has to be taken up by the intact mycoplasmas present in the medium before it can be converted into thymine.

TABLE 1
TP activity in the medium of MCF-7/HYOR cell cultures
(% conversion of nucleoside to the free pyrimidine base)
or in the presence of 0.025 U of recombinant E. coli
TP. Values are presented as means ± S.E.M. of at
least three independent experiments.

| Time | dThd | FdUrd | TFT | 5′DFUR |
|---|---|---|---|---|
| | Percent conversion of nucleoside | | | |
| | in MCF-7/HYOR medium | | | |
| 2 hours | 71 | 43 | 8 | 5 |
| 16 hours | 97 | 77 | 55 | 22 |
| | Recombinant E. coli TP | | | |
| 2 hours | 82 | 57 | 48 | 26 |
| 16 hours | 93 | 63 | 85± | 64 |

### Example 5

### Cytostatic Activity of Nucleoside Analogues in Combination with Mycoplasma Antibiotics or Inhibitors of Mycoplasma Nucleoside or Nucleotide Metabolising Enzymes

The cytostatic activity of 5′DFUR, TFT, FdUrd, CIdUrd, BrdUrd, and IdUrd was determined in both MCF-7 and MCF-7/HYOR cell cultures in the absence or presence of TPi (Table 2). With the exception of 5′DFUR, the cytostatic activity of the nucleoside analogues was 20- to 150-fold lower in the infected MCF-7/HYOR cell cultures compared to control MCF-7 cells. The decreased cytostatic activity of the nucleoside analogues observed in the MCF-7/HYOR cell cultures could be completely restored by co-administration of TPi (10 μM) (Table 2). These results indicate that M. hyorhinis-encoded TP converts the pyrimidine nucleoside analogues into their respective pyrimidine bases, resulting in a decreased cytostatic activity of these compounds. In contrast, 5′DFUR was markedly more cytostatic in infected MCF-7/HYOR cells, indicating that the mycoplasma-encoded TP efficiently converted this prodrug into 5FU. The IC₅₀ values of the parent compound 5FU were not significantly different in MCF-7 and MCF-7/HYOR cell cultures. This is obviously due to the TP-independent conversion of 5FU to its active metabolite (FdUMP).

TABLE 2
Cytostatic activity of pyrimidine nucleoside analogues against M. hyorhinis-
infected and uninfected MCF-7 cells in the presence or absence of TPi. Values are
presented as means ± S.E.M. of at least three independent experiments.

| | IC₅₀^{a} (μM) | | | | | |
|---|---|---|---|---|---|---|
| | MCF-7 | | | MCF-7/HYOR | | |
| | As such | +TPi (10 μM) | Ratio^{b} | As such | +TPi (10 μM) | Ratio^{b} |
| Compound | (1) | (2) | (1)/(2) | (1) | (2) | (1)/(2) |
| FdUrd | 0.003 ± 0.002 | 0.003 ± 0.002 | 1.0 | 0.42 ± 0.18 | 0.003 ± 0.001 | 140 |
| TFT | 0.39 ± 0.12 | 0.21 ± 0.11 | 1.8 | 6.0 ± 3.19 | 0.18 ± 0.07 | 33 |
| CldUrd | 0.76 ± 0.19 | 0.64 ± 0.15 | 1.2 | 13 ± 2.87 | 1.4 ± 0.70 | 9.3 |
| BrdUrd | 0.59 ± 0.10 | 0.36 ± 0.01 | 1.6 | 8.6 ± 1.17 | 0.84 ± 0.24 | 10 |
| IdUrd | 1.1 ± 0.26 | 0.98 ± 0.39 | 1.1 | 12 ± 0.5 | 0.31 ± 0.05 | 39 |
| 5FU | 0.81 ± 0.24 | 0.62 ± 0.29 | 1.3 | 0.75 ± 0.24 | 0.53 ± 0.25 | 1.4 |
| 5′DFUR | >100 | >100 | >1< | 3.5 ± 0.53 | >100 | <0.035 |

^{a}50% Inhibitory concentration, or compound concentration required to inhibit tumor cell proliferation by 50%.
^{b}The ratio (1)/(2) represent the ratio of IC₅₀ in the absence of TPi to the IC₅₀ in the presence of TPi.

### Example 6

### Combinations of Anti-Cancer Drugs and Plasmocin

The cytostatic activity of TFT, FdUrd, BrdUrd, 5′DFUR, and 5FU was also investigated in the presence of the antibiotic plasmocin (25 μg/ml), which was added to the MCF-7 and MCF-7/HYOR cells one day or three days before addition of the test compounds (Table 3). Addition of plasmocin to the MCF-7 cells did not alter the IC₅₀ values of the test compounds (data not shown). However, pre-incubation of the MCF-7/HYOR cell cultures with the antibiotic for one day partially restored the decreased cytostatic activity of the test compounds, while three days pre-incubation with plasmocin completely restored the anti-proliferative activity of TFT, FdUrd and BrdUrd. Whereas plasmocin did not affect the activity of 5FU, 5′DFUR lost its cytostatic activity in MCF-7/HYOR cell cultures pre-treated with plasmocin.

TABLE 3
Cytostatic activity of pyrimidine nucleoside analogues against M. hyorhinis-
infected MCF-7 and uninfected MCF-7 cells, pretreated with plasmocin for one day or
three days prior to addition of the test compounds.

| | IC₅₀^{a} (μM) | | | |
|---|---|---|---|---|
| | MCF-7/HYOR | | | |
| | | +plasmocin (25 μg/ml) 1 | +plasmocin (25 μg/ml) 3 | |
| | | day prior to addition of test | days prior to addition of test | MCF-7 |
| Compound | As such | compounds | compounds | As such |
| TFT | 6.0 ± 3.19 | 0.45 ± 0.09 | 0.19 ± 0.06 | 0.39 ± 0.12 |
| BrdUrd | 8.6 ± 1.17 | 2.22 ± 1.1 | 0.74 ± 0.2 | 0.59 ± 0.10 |
| FdUrd | 0.42 ± 0.18 | 0.018 ± 0.0022 | 0.003 ± 0.001 | 0.003 ± 0.002 |
| 5FU | 0.75 ± 0.24 | 0.74 ± 0.11 | 0.67 ± 0.11 | 0.81 ± 0.24 |
| 5′DFUR | 3.5 ± 0.53 | >100 | >100 | >100 |

^{a}50% Inhibitory concentration or compound concentration required to inhibit tumor cell proliferation by 50%. IC50s are determined as known in the art. The anti-cancer compounds are tested at the following concentrations: 250 μM, 50 μM, 10 μM, 2 μM, 0.4 μM and 0.08 μM in order to determine the IC₅₀s.
Values are presented as means ± S.E.M. of at least three independent experiments.

### Example 7

### Metabolism and Incorporation of Pyrimidine Nucleoside Analogues into Nucleic Acids

Most pyrimidine nucleoside analogues are cytostatic because they inhibit DNA and/or RNA synthesis by inhibiting thymidylate synthase and/or by being incorporated into the nucleic acids of tumor cells. The incorporation of dThd, BrdUrd, TFT and dUrd into nucleic acids was respectively 85-, 45-, 40- and 3-fold reduced in infected MCF-7/HYOR cells in comparison with uninfected MCF-7 cells (FIG. 4). Addition of TPi to the radiolabeled drug-exposed MCF-7/HYOR cell cultures fully restored the impaired incorporation to normal levels. These results show that M. hyorhinis-encoded TP markedly prevents the conversion of the drugs to their active metabolites, presumably by releasing the free pyrimidine base and thus by preventing proper anabolism of the pyrimidine nucleoside analogues to their phosphorylated metabolites. There was no difference in the incorporation of the free pyrimidine bases thymine, uracil, 5FU and TF-thymine into nucleic acids between the infected and uninfected MCF-7 cells. Interestingly, the incorporation of these pyrimidine bases was very small, presumably by poor, if any, TP-induced conversion to their respective nucleoside derivatives.

Unlike what may have been expected from the cell proliferation data, M. hyorhinis infection did not affect the incorporation of FdUrd into nucleic acids. FdUrd elicits its cytostatic activity by inhibition of thymidylate synthase as its 5′-monophosphate derivative FdUMP. The formation of phosphorylated FdUrd metabolites was therefore investigated and compared with the metabolites of dThd, BrdUrd and TFT (Table 4). In MCF-7/HYOR cells, low, if any significant levels of di- and triphosphate derivatives of dThd, BrdUrd, FdUrd and TFT were detected. However, in the presence of TPi, the levels of TFT-5′-monophosphate were increased by 2.7-fold, whereas FdUrd 5′-monophosphate levels were markedly increased by 18-fold. These data are strongly suggestive for TS as the main mechanism of cytostatic action of FdUrd whereas the other drugs, including TFT, may predominantly exert their cytostatic activity upon incorporation into nucleic acids. In the presence of TPi, almost all dThd or BrdUrd was incorporated into nucleic acids while 66% of the TFT but almost no FdUrd was incorporated into the nucleic acids. This is obviously due to the fact that dThd and BrdUrd are much better substrates for cellular TK than TFT and FdUrd. The data in Table 4 again confirm the degradation of all nucleosides to their inactive bases in MCF-7/HYOR cells, whereas administration of TPi to the cell cultures inhibits this catabolic activity.

TABLE 4
Percent of drug-derived radiolabel (i.e. from TFT, FdUrd, BrdUrd and dThd)
added to MCF-7/HYOR cell cultures. Values are presented as means of at least three
independent experiments. The S.E.M. are not shown but are less than 5% of the values.

| | % of drug-derived radiolabel in MCF-7/HYOR cell cultures | | | | | | |
|---|---|---|---|---|---|---|---|
| | | | In the cytosol of the cells | | | | Incorporatio |
| | In medium | | Nucleoside/ | 5′-mono- | 5′-di- | 5′-tri- | into DNA/ |
| Compound | Base | Nucleoside | nucleobase | phosphate | phosphate | phosphate | RNA |
| TFT | 56.7 | 23.3 | 11.0 | 6.1 | 0.6 | 0.6 | 1.8 |
| TFT + TPi | 0.0 | 6.4 | 8.6 | 16.7 | 2.1 | 0.8 | 65.4 |
| FdUrd | 49.5 | 33.2 | 11.6 | 3.5 | 0.9 | 0.3 | 1.0 |
| FdUrd + TPi | 5.3 | 11.4 | 18.1 | 62.9 | 0.8 | 0.6 | 0.9 |
| BrdUrd | 49.5 | 42.0 | 6.4 | 0.4 | 0.2 | 0.2 | 1.4 |
| BrdUrd + TPi | 0.1 | 0.9 | 2.7 | 1.1 | 0.4 | 0.4 | 94.5 |
| dThd | 74.6 | 11.5 | 7.5 | 0.6 | 0.3 | 0.2 | 5.3 |
| dThd + TPi | 0.7 | 0.5 | 1.3 | 0.3 | 0.1 | 0.2 | 97.0 |

### Example 8

### In Vivo Experiments—Antitumor Activity of Nucleoside Analogues in Combination with Mycoplasma Antibiotics or Inhibitors Of Mycoplasma Nucleoside or Nucleotide Metabolising Enzymes

### a) Establishment of the Animal Model

### Cell Cultures.

FM3A cells are grown in Dulbecco's modified minimum essential medium (DMEM, Life Technologies, Inc., Rockville, Md.) supplemented with 10 mM Hepes (Life Technologies, Inc., Rockville, Md.) and 10% fetal bovine serum (FBS, Harlan Sera-Lab Ltd., Loughborough, UK). The cells are infected with Mycoplasma hyorhinis by adding “infected” medium to the cell cultures. The presence of M. hyorhinis in FM3A cell cultures is confirmed by a species-specific PCR.

### Animals.

Female severe combined immunodeficient (SCID) mice, weighing about 20 g are used for all experiments. The animals are bred at the animal facility of the K.U.Leuven.

### Animal Experiments.

FM3A cells infected with Mycoplasma hyorhinis (10.10⁶ or 2.10⁶ cells/200 μl DMEM without serum) are injected intraperitoneally in SCID mice. At different time points after inoculation of the cells, mice are dissected and tumors, ascites fluid, blood en several organs are collected. DNA is extracted from the collected samples and processed for PCR analysis to verify the presence of M. hyorhinis.

Experiments with nucleoside- or nucleotide-based anti-cancer drugs in combination with (a) mycoplasma antibiotics or (b) inhibitors of mycoplasma nucleoside or nucleotide metabolising enzymes are administered to the animals and the tumor growth and volume is measured.

### Example 9

### Cytostatic Activity of Cytidine Analogues in Combination With an Inhibitor of Thymidine Phosphorylase

The cytostatic activity of cytarabine (araC) and gemcitabine was determined in both MCF-7 and MCF-7/HYOR cell cultures in the absence or presence of TPi (Table 5). The cytostatic activity of the cytidine analogues was about 14 to about 20-fold lower in the infected MCF-7/HYOR cell cultures compared to control MCF-7 cells. The decreased cytostatic activity of the cytidine analogues observed in the MCF-7/HYOR cell cultures could be restored by co-administration of TPi (10 μM) (Table 5).

TABLE 5
Cytostatic activity of cytidine analogues against M. hyorhinis-infected and
uninfected MCF-7 cells in the presence or absence of TPi. Values are presented as
means ± S.E.M. of at least three independent experiments.

| | IC₅₀^{a} (μM) | | | | | |
|---|---|---|---|---|---|---|
| | MCF-7 | | | MCF-7/HYOR | | |
| | As such | +TPi (10 μM) | Ratio^{b} | As such | +TPi (10 μM) | Ratio^{b} |
| Compound | (1) | (2) | (1)/(2) | (1) | (2) | (1)/(2) |
| araC | 0.050 ± 0.004 | 0.048 ± 0.009 | 1.0 | 1.2 ± 0.8 | 0.068 ± 0.003 | 17 |
| gemcitabine | 0.0073 ± 0.0036 | 0.0063 ± 0.0022 | 1.2 | 0.080 ± 0.028 | 0.0058 ± 0.0012 | 14 |

^{a}50% Inhibitory concentration or compound concentration required to inhibit tumor cell proliferation by 50%.
^{b}The ratio (1)/(2) represent the ratio of IC₅₀ in the absence of TPi to the IC₅₀ in the presence of TPi.

### Example 10

### Cytostatic Activity of a Purine Analogue in Combination With an Inhibitor of Thymidine Phosphorylase

The cytostatic activity of cladribine (a purine analogue) was determined in both MCF-7 and MCF-7/HYOR cell cultures in the absence or presence of TPi (Table 6). The cytostatic activity of the nucleoside analogue was about 30-fold lower in the infected MCF-7/HYOR cell cultures compared to control MCF-7 cells. The decreased cytostatic activity of the purine analogue observed in the MCF-7/HYOR cell cultures could be restored by co-administration of TPi (10 μM) (Table 6).

TABLE 6
Cytostatic activity of a purine nucleoside analogue against M. hyorhinis-infected
and uninfected MCF-7 cells in the presence or absence of TPi. Values are presented
as means ± S.E.M. of at least three independent experiments.

| | IC₅₀^{a} (μM) | | | | | |
|---|---|---|---|---|---|---|
| | MCF-7 | | | MCF-7/HYOR | | |
| | As such | +TPi (10 μM) | Ratio^{b} | As such | +TPi (10 μM) | Ratio^{b} |
| Compound | (1) | (2) | (1)/(2) | (1) | (2) | (1)/(2) |
| cladribine | 0.46 ± 0.15 | 0.39 ± 0.16 | 1.2 | 13 ± 3.0 | 0.78 ± 0.23 | 17 |

^{a}50% Inhibitory concentration, or compound concentration required to inhibit tumor cell proliferation by 50%.
^{b}The ratio (1)/(2) represent the ratio of IC₅₀ in the absence of TPi to the IC₅₀ in the presence of TPi.

### Example 11

### Metabolism and Incorporation of Cytidine and Purine Nucleoside Analogues into Nucleic Acids

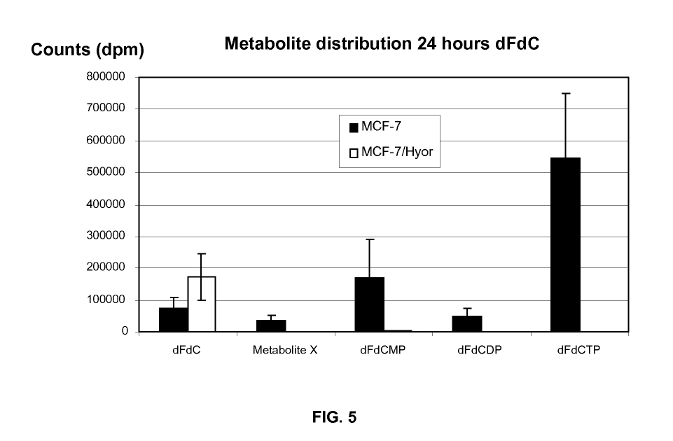

The cytostatic activity of gemcitabine (dFdC) was reduced 14-fold in MCF-7/HYOR compared to MCF-7 cells (Table 5). Therefore, the distribution of different metabolites of dFdC was investigated in these cell lines. In MCF-7 cells, gemcitabine is readily activated (phosphorylated) into dFdC monophosphate, (dFdC diphosphate) and in particular to dFdC triphosphate. In contrast, in M. hyorhinis infected MCF-7 cells, gemcitabine is still present as such 24 h after its addition and no active metabolite can be detected (see FIG. 5). These data indicate that mycoplasma infection inhibits the activation, and consequently cytostatic activity, of dFdC.

The present inventors have found that unexpectedly TPi combined with gemcitabine (a cytidine analogue) and cladribine (a purine analogue) reverse the damaging effect of mycoplasmas against these anticancer drugs and fully restored the cytotoxicity of these drug against cancer.

Indeed, both gemcitabine and cladribine are drugs that are not expected to be substrates for TP because they belong to two entirely different classes of compounds for which so far, it has never been shown that they are sensitive to the degradation by TP. The TP enzyme itself has only been shown to act on thymidine and deoxyuridine analogues, never on cytidine and adenosine (purine) analogues.

### Example 12

### Combinations of Cytosine- or Purine-Based Anti-Cancer Drugs and Plasmocin

The cytostatic activity of gemcitabine, cladribine and cytarabine is also investigated in the presence of the antibiotic plasmocin (25 μg/ml), which is added to the MCF-7 and MCF-7/HYOR cells one day or three days before addition of the test compounds. Addition of plasmocin to the MCF-7 cells does not alter the IC₅₀ values of the test compounds. However, pre-incubation of the MCF-7/HYOR cell cultures with the antibiotic for one day partially restores the decreased cytostatic activity of the test compounds, while three days pre-incubation with plasmocin restores the anti-proliferative activity of gemcitabine, cladribine and cytarabine.

### Example 13

### Combinations of Cytosine- or Purine-Based Anti-Cancer Drugs and Doxycycline

The cytostatic activity of gemcitabine, cladribine and cytarabine is also investigated in the presence of the antibiotic doxycycline, which is added to the MCF-7 and MCF-7/HYOR cells one day or three days before addition of the test compounds. Addition of doxycycline to the MCF-7 cells does not alter the IC₅₀ values of the test compounds. However, pre-incubation of the MCF-7/HYOR cell cultures with the antibiotic for one day partially restores the decreased cytostatic activity of the test compounds, while three days pre-incubation with doxycycline restores the anti-proliferative activity of gemcitabine, cladribine and cytarabine.

1. A combination of therapeutic agents comprising:
(a) a cytosine-based anti-cancer drug and/or a purine-based anticancer drug, and
(b) a therapeutic agent selected from the group consisting of thymidine phosphorylase inhibitors, and antibiotics against Mollicutes bacteria,
provided that when said cytosine-based anti-cancer agent is gemcitabine, the antibiotic against Mollicutes bacteria is not ciprofloxacin.

2. The combination according to claim 1, wherein said cytosine-based anti-cancer drug is selected from the group consisting of cytarabine, gemcitabine, troxacitabine, sapacitabine.

3. The combination according to claim 1, wherein said purine based anti-cancer drug is selected from 6-thioguanine, 6-mercaptopurine, azathioprine, 2-chloroadenine, 2-fluoroadenine, nelarabine, 2′,2′-difluoroguanosine, 9β-D-arabinosylguanine (araG), clofarabine, cladribine, 6-methyl-purine-riboside, and fludarabine.

4. The combination according to claim 1, wherein said therapeutic agent (b) is an uracil derivative, a solvate or a pharmaceutically acceptable salt thereof, said uracil derivative being represented by the structural formula (I) 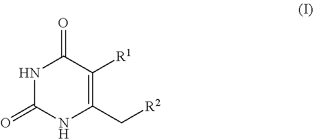
wherein:
R¹ is selected from chloro, bromo, iodo, cyano or C₁₋₄alkyl; R² is a 4-8 membered heterocyclic group having 1, 2 or 3 nitrogen atoms, optionally substituted by one or more substituents independently selected from the group consisting of C₁₋₄alkyl, imino, hydroxyl, hydroxymethyl, methanesulfonyloxy, amino and nitro; or R² is an amidinothio group, the nitrogen atoms of which may each be independently substituted by C₁₋₄alkyl; or R² is a guanidino group, the nitrogen atoms of which may each be independently substituted by C₁₋₄alkyl or cyano; or R² is C₁₋₄alkyl-amidino; or R² is amino, mono-C₁₋₄alkylamino or di-C₁₋₄alkylamino; or R² is a group with the structural formula —CH₂N(R^{a})R^{b} wherein R^{a} and R^{b} are independently hydrogen or C₁₋₄alkyl or R^{a} and R^{b} may form a pyrrolidine ring together with the nitrogen atom to which they are bonded; or R² is a group with the structural formula —NH—(CH₂)ₘ—Z wherein Z is cyano, amino, mono-C₁₋₄alkylamino or di-C₁₋₄alkylamino, and m is an integer from 0 to 3; or R² is a group with the structural formula NR^{c}(CH₂)ₙ—OH in which R^{c} is hydrogen or C₁₋₄alkyl, and n is an integer from 1 to 4; or R² is a group with the structural formula—X—Y in which X is S or NH, and Y is selected from the group consisting of 2-imidazolin-2-yl, 2-imidazolyl, 1-methylimidazol-2-yl, 1,2,4-triazol-3-yl, 2-pyrimidyl and 2-benzimidazolyl group; or R² is a ureido or thioureido group, the nitrogen atoms of which may each be independently substituted by C₁₋₄alkyl.

5. The combination according to claim 4, wherein in said structural formula (I) R² is selected from the group consisting of 2-iminopyrrolidin-1-yl, 1-azetidinyl, 1-pyrrolidinyl, 2-pyrrolin-1-yl, 3-pyrrolin-1-yl, 1-pyrrolyl, 1-pyrazolidinyl, 2-pyrazolin-1-yl, 3-pyrazolin-1-yl, 4-pyrazolin-1-yl, 1-pyrazolyl, 1-imidazolidinyl, 2-imidazolin-1-yl, 3-imidazolin-1-yl, 4-imidazolin-1-yl, 1-imidazolyl, 1,2,3-triazol-1-yl, 1,2,4-triazol-1-yl, piperidino, 1-piperazyl, morpholino, 1-perhydroazepinyl, 1-perhydroazocinyl, amidino-thio, N-methylamidinothio, N,N′-dimethylamidinothio, 1-guanidino, 1-methylguanidino, 3-methylguanidino, 2,3-dimethylguanidino, 2-cyano-3-methylguanidino, acetoamidino, N-methylamino, N,N-dimethylamino, N-ethylamino, N,N-diethylamino, N-propylamino, N-isopropylamino, N-methylaminomethyl, N,N-dimethylaminomethyl, 1-pyrrolidinylmethyl, N,N-dimethylhydrazino, N-(2-aminoethyl)amino, N-(2-(N,N-dimethyl)amino-ethyl)amino, N-(3-aminopropyl)amino, N-(2-cyanoethyl)amino, N-(2-hydroxyethyl)-N-methylamino, N-(3-hydroxypropyl)amino, N-(4-hydroxy-butyl)amino, 2-imidazolin-2-thio, 2-imidazolin-2-amino, imidazol-2-thio, 1-methylimidazol-2-thio, 1,2,4-triazol-3-thio, pyrimidin-2-thio, benzimidazol-2-thio and 3-methylthioureido.

6. The combination according to claim 4 or claim 5, wherein in said structural formula (I), R¹ is bromo, cyano or methyl.

7. The combination according to claim 4 or 5, wherein said uracil derivative, a solvate or a pharmaceutically acceptable salt thereof is selected from the group consisting of 5-chloro-6-(1-[2-imino-pyrrolidinyl]methyl)uracil hydrochloride, 6-imidazolylmethyl-5-fluorouracil, 5-chloro-6-(1-pyrrolidinylmethy)uracil, 5-bromo-6-(1-pyrrolidinylmethyl)uracil, 5-chloro-6-(1-azetidinylmethyl)-uracil, 5-bromo-6-(1-(2-iminopyrrolidinyl)methyl)uracil hydrochloride, 5-cyano-6-(1-(2-iminopyrrolidinyl)methyl)uracil, 5-chloro-6-(1-(2-imino-imidazolidinyl)methyl)uracil, 5-bromo-6-(1-(2-iminoimidazolidinyl)-methyl)uracil, 5-chloro-6-(1-imidazolylmethyl)uracil hydrochloride, 2-(5-chlorouracil-6-ylmethyl)isothiourea hydrochloride, 2-(5-cyanouracil-6-ylmethyl)isothiourea hydrochloride and 5-chloro-6-(1-guanidino)methyl-uracil hydrochloride.

8. The combination according to claim 1, wherein said therapeutic agent (b) is selected from the group consisting of thymidine phosphorylase inhibitors, and wherein the molar ratio between said cytosine or purine-based anti-cancer drug (a) and said therapeutic agent (b) ranges from 25:1 to 0.01:1.

9. The combination according to claim 1, being a combination of 5-chloro-6-(1-[2-imino-pyrrolidinyl]methyl)uracil hydrochloride, with a cytosine- or purine-based anti-cancer drug (a) selected from the group consisting of cytarabine, gemcitabine, troxacitabine, sapacitabine, 6-thioguanine, 6-mercaptopurine, azathioprine, nelarabine, 2-chloroadenine, 2-fluoroadenine, 2′,2′-difluoroguanosine, 9-β-D-arabinosylguanine (araG), clofarabine, cladribine, 6-methyl-purine-riboside, and fludarabine.

10. The combination according to claim 1, wherein said antibiotic against Mollicutes bacteria is selected from the group consisting of plasmocin; herbicolin A; tetracyclines including doxycycline or minocycline; (fluoro)quinolones including ciprofloxacin, enrofloxacin, gemifloxacin or levofloxacin; macrolides including azithromycin, erythromycin or clarithromycin; and linkomycin.

11. The combination according to claim 1, wherein the molar ratio between said cytosine- or purine-based anti-cancer drug and said antibiotic against Mollicutes bacteria ranges from 10:1 to 0.01:1.

12. The combination according to claim 1, wherein said antibiotic against Mollicutes bacteria is a Mycoplasma-specific antibiotic.

13. A pharmaceutical composition comprising one or more pharmaceutically acceptable carriers or excipients and, as active ingredient, a therapeutically effective amount of the combination of therapeutic agents according to claim 1.

14. A method for the prevention or treatment of cancer in an animal comprising providing and/or administering to an animal in need thereof a therapeutically effective amount of the combination according to claim 1, or a pharmaceutical composition according to claim 13.

15. The method according to claim 14, comprising the consecutive administration of the therapeutic agents, wherein the therapeutic agent (b) is administered prior to the cytosine- or purine-based anticancer drug.

16. The method according to claim 15, wherein said therapeutic agent (b) is administered from 1 to 4 days prior to said cytosine- or purine-based anticancer drug (a).
